(19) United States
(12) Patent Application Publication
(10) Pub. No.: US 2010/0093626 A1
(43) Pub. Date: Apr. 15, 2010

(54) PEPTIDE AND SMALL MOLECULE AGONISTS OF EPHA AND THEIR USES IN DISEASE

(76) Inventor: Bingcheng Wang, Solon, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/519,294

(22) PCT Filed: Dec. 14, 2007
(86) PCT No.: PCT/US2007/087613

§ 371 Date: Jun. 15, 2009

Related U.S. Application Data
(60) Provisional application No. 60/875,096, filed on Dec. 15, 2006.

(51) Int. Cl. A61K 38/16; A61K 31/4355; A61P 35/00

(52) U.S. Cl. 514/12; 514/291

(57) ABSTRACT

A method of treating a neoplastic disorder in a subject includes administering to a Eph kinase expressing neoplastic cell of the subject being treated an EphA agonist and at least one immunosuppressant.

### RELATED APPLICATIONS

This application claims priority from Provisional Application No. 60/875,096, filed Dec. 15, 2006, the subject matter which is incorporated herein by reference in its entirety.

### GOVERNMENT FUNDING

This invention was made with government support under Grant No. NIH Grant No, R01 CA96533, CA92259 and DOD Grant No. DAMD17-99-1-9019 awarded by the National Institutes of Health. The United States government has certain rights in the invention.

### BACKGROUND

Prostate cancer is the second leading cause of cancer death for North American men, and is known to exhibit widely variable aggressiveness. While the majority (80%) of 220,000 newly diagnosed prostate cancer cases each year will remain dormant and do not metastasize during the lifetime of the patients, over 30,000 of them will rapidly metastasize and kill the patients. The molecular mechanisms governing the, slow, non-threatening progression versus the rapid emergence of metastasizing lethal disease, is unclear. It is suggested that the ability to block cytokine-induced prostate cancer cell migration may potentially render these cells stationary and non-metastatic.

### SUMMARY OF THE INVENTION

The present invention relates to a method of treating a neoplastic disorder in a subject. The method includes administering to an Eph kinase expressing neoplastic cell of the subject being treated an EphA agonist and at least one immunosuppressant. The administration of the EphA agonist can modulate (e.g., activate) an Eph kinase in the cell of the subject.

In an aspect of the invention the neoplastic disorder can include at least one of lung cancer, brain cancer, prostate cancer or breast cancer. The cancer can be associated with expression of Eph kinase in the cancerous cells.

In another aspect of the invention, the immunosuppressant can include an mTOR inhibitor. The mTOR inhibitor can include sirolimus and/or a sirolimus analog.

In a further aspect of the invention, the Eph agonist can be an ephrin. The ephrin can include ephrin-A1, ephrin A5, or a combination thereof. The EphA agonist can have a cooperative effect with the mTOR inhibitor.

The present invention also relates to a method of treating cancer in a subject. The method includes administering to an EphA kinase expressing cancer cell of a subject being treated a therapeutically effective amount of an EphA agonist and an mTOR inhibitor. The administration of the EphA agonist can modulate an EphA kinase in the cancer cell of the subject.

In an aspect of the invention, the cancer can include at least one of lung cancer, brain cancer, prostate cancer or breast cancer. The mTOR inhibitor can include sirolimus or a sirolimus analog. The EphA agonist can be an ephrin. The ephrin can include ephrin-A1, ephrin A5, or a combination thereof. The EphA agonist can have a cooperative effect with the mTOR inhibitor.

The present invention further relates to a pharmaceutical composition for treating a neoplastic disorder in a subject. The pharmaceutical composition includes a therapeutically effective amount of an EphA agonist and an mTOR inhibitor. The mTOR inhibitor can include sirolimus or a sirolimus analog. The EphA agonist can be an ephrin. The ephrin can include ephrin-A1, ephrin A5, or a combination thereof. The EphA agonist can have a cooperative effect with the mTOR inhibitor.

### BRIEF DESCRIPTION OF THE DRAWINGS

FIG. 1. illustrates that the activation of endogenous EphA2 kinase in DU145 cells negatively regulates Akt and ERK1/2 activities, which was enhanced by EphA2 overexpression. A. Immunoblot analysis of the expression and tyrosine phosphorylation of EphA2 in DU145 cells. Parental DU145 cells were stably infected with a retroviral vector expressing wild-type EphA2 or empty vector. Clonal puromycin-resistant cells were isolated and expanded. The selected cells at 70-80% confluence were stimulated with 2 μg/ml ephrin-A1-Fc for the indicated times. The total cell lysates were resolved by SDS-PAGE and then probed with rabbit polyclonal anti-p-EphA/B and anti-EphA2 antibodies. Similar loading was indicated by reprobing the membrane with a mouse monoclonal anti-a-tubulin antibody. B. Activation of EphA2 by its ligand leads to the downregulation of Akt and ERK1/2 kinase activities. The cells were treated as described in (A). Cell lysates were subject to immunoblot with the indicated antibodies.

FIG. 2 illustrates that pretreatment with okadaic acid prevented Akt dephosphorylation induced by ephrin-A1. DU145-vector cells were pretreated with okadaic acid (10, 50, and 250 nM) for 1 h, followed by stimulation with ephrin-A1-Fc for 30 min. The total cellular lysates were prepared and subjected to immunoblot analyses as described in FIG. 1 using the indicated antibodies. Notes: Okadaic acid inhibits only PP2A at low concentrations (<100 nM), but it inhibits both PP1 and PP2A at higher concentrations (>100 nM).

FIG. 3 illustrates that overexpression of EphA2 inhibits cell growth and sensitizes cells to serum withdrawal in vitro. A. DU145 cells were transiently transfected with plasmids encoding wild-type EphA2 or empty vector. The resultant transfectants were plated into 35-mm dishes and selected for 2 weeks in complete medium containing 1 μg/ml puromycin. Cells were fixed with 4% paraformaldehyde for 20 min and then stained with 0.5% crystal violet in methanol for 20 min. Colonies (>1.0 mm) were visually scored independently by two different researchers. Cotransfection with a β-galactosidase-expressing vector followed by X-gal staining was used to gauge transfection efficiency. B. Cells were plated onto 12-well plates at the density of 2×103 cells/well and grown in complete medium overnight before treatment with Fc or ephrin-A1-Fc. For growth factor withdrawal experiments, cells were cultured in RPMI1640 containing 0.5% FBS. Culture medium containing the indicated drugs was changed every 5 days. Cellular growth in medium containing 10% and 0.5% FBS was stopped after 8 and 18 days, respectively, fixed with 4% paraformaldehyde and stained with 0.5% crystal violet. C. Cells were plated onto 6-well plates at the density of 5×103 cells/well and grown in the same conditions as mentioned above. Cells were collected with trypsin-EDTA every 24 h, stained with Trypan blue, and counted using a hemocytometer. D. Cells were starved for 24 h and stimulated with 2 μg/ml of ephrin-A1-Fc for 10 and 30 min. Total cellular lysates were analyzed by immunoblot with the indicated antibodies. The expression of the a-tubulin was used as a loading control.

FIG. 4 illustrates that overexpression of EphA2 in DU145 cells enhances the growth inhibition induced by rapamycin in vitro. A. Cells were treated with rapamycin (1 nM) in the absence or presence of ephrin-A1 (2 μg/ml) for the indicated times. Immunoblot analysis was performed as described above. B. Cells were plated onto 6-well culture plates at the density of 2×103 cells/well and grown in complete medium overnight before treatment with ephrin-A1-Fc (2 μg/ml), rapamycin (10 nM) alone or in combination. Cell growth was stopped after 14 days, and cells were fixed with 4% paraformaldehyde, followed by staining with 0.5% crystal violet in methanol for 20 min. C. Cells were plated onto 6-well culture plates at the density of 5×103 cells/well and grown overnight before treatment with ephrin-A1-Fc (2 μg/ml), rapamycin (10 and 100 nM) alone or in combination. Fc or DMSO was used as control treatment. Cells were collected with trypsin-EDTA every 24 h, stained with Trypan blue, and counted using a hemocytometer.

FIG. 5 illustrates that overexpression of EphA2 suppresses growth of DU145 xenografts in vivo. A. A total of 2×106 cells were injected subcutaneously in the hind flanks of nude mice. Tumor sizes were measured twice weekly, and tumor volume (mm3) is shown at the indicated time points. The graph shows average tumor volumes±SEM in groups of 6 mice. B. H&E histological analysis of primary prostate tumors from nude mice bearing DU145-vector (left) and DU145-EphA2 (right) xenografts (Magnification, ×200). C. Immunoblot analysis of EphA2, p-Akt (Ser473), and p-ERK1/2 in DU145 xenografts as described above. Lower panel shows quantitative analysis of the relative intensities of the bands. D. Immunofluorescence staining of Ki-67 expression in the xenografts. Representative microphotographs are shown in left panel (Magnification, 200×). Right panel is a quantitative analysis showing significant difference (P=0.00139) in proliferative indices between DU145-vector and DU145-EphA2 xenografts. Data represent mean±SD, n=10 fields, for each group.

FIG. 6A illustrates selective Eph kinase and ephrin ligand interactions. The ligand specifiticy of Eph A9, EphA10, and EphB5 are not clear. Note the promiscuity of Eph-ephrin interactions within each subfamily. Cross family interactions are indicated by dotted lines. EphB5 and EphA9 are not present in humans. EphA2 and ephrin-A1 are a major Eph/ephrin pair expressed in murine lung, which is the focus of this proposal.

FIG. 6B illustrates the domain structure of Eph kinases and ephrins, and some of their interacting proteins. Glob., Globular domain; Cys., Cysteine-rich domain; FNIII, fibronectin type III repeat; J.M. juxtamembrane domain; SAM, sterile a motif; P, tyrosine phosphorylation sites.

FIG. 7 illustrates disruption of EphA2 caused increased susceptibility to skin carcinogenesis. EphA2 knockout mice on C57B6/129 mixed genetic background were backcrossed to FVB/N. Female N5 mice at age of 8 weeks were initiated with 25 μg DMBA and treated twice weekly with 100 μg/mouse TPA. Tumor number and sizes were measured weekly. Tumor multiplicity (A) and incidence (B) were plotted against time. (C) Tumor growth rate was monitored by the number of tumors larger than 4 mm in diameter. (D) Images of representative mice at week 14. The experiment was terminated at week 16 due to tumor burden.

FIG. 8 illustrates that chemically-skin tumors show accelerated malignant progression in vivo. H&E staining of papilloma from wild type mouse (A) and an invasive squamous cell carcinoma in a EphA2 knockout mouse (B). Pan-keratin staining shows tumor cells in a benign papilloma (C) and invasive squamous cell carcinoma cells infiltrating into stromal tissues (D). (E) Quantitative analysis of frequency of malignant progression in tumors from three different genotypes.

FIG. 9 illustrates EphA2-null mice developed spontaneous lung tumors. A,B) Lungs were dissected from 14 month-old mice of the indicated genotype. White arrow points to a spontaneous lung tumor, which was confirmed histologically by H&E staining (C) D,E) EphA2 is expressed in adult mouse lung as indicated by X-gal staining of β-geo driven by EphA2 promoter. Higher level expression is seen in bronchioles (arrow) than alveolar pneumocytes. Wild type lung was used as negative control (F) which has no staining.

FIG. 10 illustrates that EphA2-null mice became susceptible to urethane-induced lung carcinogensis. A) Image of freshly dissected and inflated lungs 20 weeks post urethane exposure. Differences in colors was caused by differences in degree of perfusion. B) Quantitative analyses of lung tumor multiplicity. EphA2^{-/-} mice developed significantly more tumors than wild type and heterozygous littermates (p<0.001). C) H&E histological analysis of mouse lung cancer. Genotypes are indicated in the upper right corner. a,b) adenomas, 60; c) epithelial hyperplasia (EH) near the bronchiole/alvelolar boundary, 200×; d) atypical adenomatous hyperplasia (AAH), 60×; e) microdenoma 300×, f) intra-bronchiolar adenoma, 200×.

FIG. 11 illustrates that EphA2 was upregulated in urethane-induced lung tumors. Immunofluorescence staining on frozen sections (A,B,D,F,H) or immunohistochemical staining on paraffin-embedded sections (C,E,I) was performed with an antibody raised against the ectodomain of EphA2. X-gal staining on frozen sections was used to detect reporter cassette expression (G). T, tumor tissues; N, normal lung. (A-E) Preneoplastic and neoplastic lesions from wild type mice showing EphA2 overexpression in urethane-induced microadenoma (A,B), AAH (C), solid adenoma (D), and papillary adenoma (E). Note: microadenoma expressed much more EphA2 than the adjacent normal bronchiole (arrows) which express the highest level of EphA2 in normal lungs. In knockout mice, part of EphA2 ectodomain was fused to β-geo reporter cassette and trapped inside the cells that can be detected by antibodies against the ectodomain of EphA2 or by X-gal staining. F) A spontaneous EphA2−/−adenoma stained for EphA2-β-geo fusion protein or β-gal (G). H,I) EphA2-β-geo fusion protein is upregulated in EphA2-null adenomas induced by urethane. Note the intracellular localization pattern of the fusion protein in knockout mice (F and I) contrasts with the surface expression pattern of wild type EphA2 (A-E). J) Western blot showing EphA2 overexpression in tumor tissues compared with normal lung tissues, reduced ephrin-A1 expression, and lower degree of EphA2 activation in lung tumors in wild type animals.

FIG. 12 illustrates simultaneous inhibition of Akt and ERK1/2 kinase activities. A) Most human NSCLC cell lines express high levels of EphA2, compared with control small airway epithelial cells (SAEC). B) Examples of IHC staining for expression of EphA2 on human NSCLC specimens at 300× magnification. C) EphA2 activation by ephrin-A1 inhibits both Akt and ERK activities in a subset of NSCLC cell lines. Cells were stimulated with ephrin-A1 for 10 or 30 min. Lysates were blotted with the indicated antibodies.

FIG. 13 illustrates that A) Forced expression of EphA2 in H1975 human adenocarcinoma cell line enhanced the inhibitory effects of EphA2 activation on ERK and Akt. B,C) In Boyden chamber cell migration assay, EphA2 activation suppressed integrin-mediated cell migration toward fibronectin. EphA2 overexpression reduced basal cell motility, which was further reduced by ephrin-A1 treatment. D). Forced expression of EphA2 sensitized cells to the growth inhibitory effects of Rapamycin. Ephrin-A1 alone suppressed cell growth and cooperated with Rapamycin to further reduce cell growth. E) LKR-13, a cell line derived from a mouse lung cancer with activated Ras, were growth inhibited by ephrin-A1. Co-treatment with Rapamycin showed cooperative effects. F) EphA2 activation in LKR-13 cells inhibited both Akt and ERK activities.

FIG. 14 illustrates homing of ephrin-A1-Fc to lung tumors after systemic i.p. injection. Wild type mice were treated with urethane to induce lung tumors. Twenty-five weeks later, mice were injected i.p. with 5 μg/ml ephrin-A1-Fc or 2.5 μg/ml Fc control (Fc has about half the size of ephrin-A1-Fc). Mice were sacrificed two hours later. The frozen sections were stained with two different antihuman Fc antibodies as indicated, revealing selective homing of ephrin-A1-Fc, but not control Fc, to tumor tissues that overexpress EphA2.

FIG. 15 illustrates EphA2 kinase in the urethane-induced autochthonous mouse tumors was activated following systemic administration of ephrin-A1. A) EphA2 overexpressed in tumors was selectively activated compared with normal lungs. Tumor induction and ephrin-A1-Fc treatment were carried out as in FIG. 10. Mice were sacrificed 2 hours after i.p. injection of ephrin-A1. Surface lung tumors and adjacent normal tissues were harvested under a dissection microscope and lysed. EphA2 was precipitated, and blotted as indicated. Quantitative analysis showed the increased EphA2 activation and degradation (lower panels). B) Administration of ephrin-A1-Fc, but not Fc control, caused a dose-dependent EphA2 activation in tumors following short term treatment (two hours) of wild type mice. C) Following long-term treatment, twice a week for two weeks, EphA2 expression recovered possibly as a results of ligand-stimulated receptor expression. Note EphA2 remained activated in tumors (compare lanes 1 and 3).

FIG. 16 illustrates A) Systemic treatment with ephrin-A1-Fc, but not control Fc, inhibited the growth of H1975 NSCLC xenografts. Eight-week old NCR nude mice were injected with 2×106 H1975 cells s.c. into the hind flanks. One week later, when the tumors had reached an average of about 100 mm3, mice (5/group) were injected i.p. twice a week with Fc or ephrin-A1-Fc at 2.5 or 5.0 μg/g. Equal volume of PBS was used as control. The day of the first treatment was designated as day 1. Rapid tumor growth necessitated termination of experiment at day 18. B) Comparison between ephrin-A1-Fc and Alimta for therapeutic efficacy on H460 xenografts. Cell implantation and treatment were the same as in (A). Alimta/Pemetrexed was injected at 150 mg/kg, i.p daily×5 days), or ephrin-A1-Fc (5 μg/g, mouse, i.p, once every two days, for five times). Note ephrin-A1-Fc treatment achieved better efficacy of tumor growth inhibition than Alimta, a new drug for lung cancer therapy in this cell type. * Indicates significant difference between PBS and ephrin-A1-treated groups, p<0.05.

FIG. 17 illustrates establishment and characterization of EphA2+/+, EphA2+/− and EphA2−/− tumor cell lines. A) Immortalized mouse lung tumor cell lines with epithelial morphology (phase, top panel) were tested for the expression of β-geo reporter cassette by X-gal staining and EphA2 by immunofluorescence (IF) staining. Pankeratin IF staining was used to verify epithelial origin of the cells. B) Cells of the indicated genotype were stimulated with ephrin-A1, and total cell lysates were blotted with the indicated antibodies. Note: 1) Low basal activation of EphA2 kinase in EphA2+/+ and EphA2+/− cells. 2) Sustained inhibition of Akt activities in EphA2+/+ and EphA2+/− cells, but not in EphA2−/− cells, while ERK1/2 activities were minimally affected. The activities of S6K kinase, the downstream target of Akt, was also inhibited. The selective inhibition of Akt but not ERK activities is similar to human NSCLC with activated Ras oncogene. C) An EphA2-null cell line shows strong tumorigenecity in FVB syngeneic host. Two million cells in 100 μl PBS were injected s.c. into the hind flanks. Mice were photographed 17 days later. Arrows point to tumors.

### DETAILED DESCRIPTION

The present invention relates to methods and compositions that provide for the treatment, inhibition, and management of diseases and disorders associated with the expression or overexpression of EphA kinase (e.g., EphA1, EphA2, and EphA3) and/or cell hyperproliferative diseases and disorders. The present invention is based at least in part on the discovery that EphA kinase (e.g., EphA1, EphA2, and EphA3) can function as a tumor suppressor and that activation of EphA kinase (e.g., EphA1, EphA2, and EphA3) can inhibit cancer cell growth, migration and/or proliferation in, for example, prostate cancer, breast cancer, colorectal cancer, skin cancer, and lung cancer.

A particular aspect of the invention relates to methods and therapeutic agents or compositions containing compounds that inhibit cancer cell proliferation, invasion, and survival, particularly those cancer cells that overexpress EphA kinase or cancer cells that have a hyperactive Ras/Raf/MEK/ERK1/2 and/or PI3/AKT signaling pathway. The therapeutic agents or compounds can comprise agonists of EphA (e.g., EphA1, EphA2, and EphA3). Exemplary agonists of EphA kinase (e.g., EphA1, EphA2, and EphA3) can include peptide and small molecule EphA kinase agonists.

The present invention further relates to methods and therapeutic agents for the treatment, inhibition, or management of metastases of cancers of epithelial cell origin, especially human cancers of the breast, lung, skin, prostate, bladder, and pancreas, and renal cell carcinomas and melanomas. Other aspects of the present invention relate to methods of inhibiting proliferation of cancer cells by suppressing the Ras/Raf/MEK/ERK1/2 signaling pathway with a therapeutic agent in accordance with the present invention. Still other aspects of the present invention relate to methods of inhibiting cancer cell survival by suppressing PI3/AKT signaling pathway with a therapeutic agent in accordance with the present invention.

Further compositions and methods of the invention include other types of active ingredients in combination with the therapeutic agents of the invention. In other embodiments, the methods of the invention are used to treat, prevent or manage other diseases or disorders associated with cell hyperproliferation, for example but not limited to restenosis, inflammation, asthma, chronic obstructive lung diseases, abnormal angiogenesis (particularly in the eyes), psoriasis, etc. The present invention also relates to methods for the treatment, inhibition, and management of cancer or other hyperproliferative cell disorder or disease that has become partially or completely refractory to current or standard cancer treatment, such as chemotherapy, radiation therapy, hormonal therapy, and biological therapy.

As used herein, the term “EphA therapeutic agent” is a generic term which include any compound (agent) which regulates signaling through the EphA pathway. This compound can also suppress or inhibit signaling of the PI3/AKT pathway and/or MAPK/ERK1/2 pathway. Exemplary, EphA therapeutic agents can be used for treating cancer (tumor). In certain embodiments, EphA therapeutic agent can activate function of EphA (e.g., EphA1, EphA2, or EphA3), enhance the interaction of an EphA ligand (e.g., ephrin A1 and ephrin A5) and EphA (e.g., EphA1, EphA2, or EphA3), activate the phosphorylation of EphA (e.g., EphA1, EphA2, or EphA3), enhance dimerization of EphA (e.g., EphA1, EphA2, or EphA3), or activate any of the downstream signaling events upon binding of an EphA ligand (e.g., ephrin A1 and ephrin A5) to EphA (e.g., EphA1, EphA2, or EphA3). For example, the EphA therapeutic agents can be capable of binding to an EphA2 polypeptide (e.g., the ligand-binding domain of an EphA2 polypeptide) and function as EphA2 ligands.

Generally, the EphA therapeutic agents include any substances that act as agonists of EphA. Such EphA therapeutic agents include, but are not limited to, a protein, a peptide, a small organic molecule, a peptidomimetic, an antibody, and a nucleic acid. In an aspect of the invention, these substances can comprise exogenous or non-native peptides or small molecules that have a molecular weight of about 50 daltons to about 2,500 daltons.

In certain specific aspects, the EphA therapeutic agents of the present invention include a peptide, such as those which activate EphA kinase function. These peptides are also referred to herein as EphA agonistic peptides. These agonistic peptides can specifically target the ligand-binding domain of EphA kinase. Optionally, these peptides can suppress AKT kinase and/or MAPK/ERK1/2 kinase.

Exemplary EphA agonistic peptides can comprise about 4 to about 20 amino acids and have a molecular weight of about 600 daltons to about 2,500 daltons. The EphA agonistic peptides include, but are not limited to, EP1 (SEQ ID NO: 1), EP2 (SEQ ID NO: 2), and EP3 (SEQ ID NO: 3). In certain embodiments, an EphA agonistic peptide has an amino acid sequence that is at least 80% identical to an amino acid sequence as set forth in SEQ ID NO: 1, 2 or 3. In certain cases, the functional variant has an amino acid sequence at least 85%, 90%, 95%, 97%, 98%, 99% or 100% identical to an amino acid sequence as set forth in SEQ ID NO: 1, 2 or 3.

In certain embodiments, the present invention contemplates making functional variants by modifying the structure of an EphA agonistic peptide for such purposes as enhancing therapeutic efficacy, or stability (e.g., ex vivo shelf life and resistance to proteolytic degradation in vivo). Modified EphA agonistic peptides can be produced, for instance, by amino acid substitution, deletion, or addition. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of an EphA agonistic peptide results in a functional homolog can be readily determined by assessing the ability of the variant EphA agonistic peptide to produce a response in cells in a fashion similar to the wild-type EphA agonistic peptide.

The present invention further contemplates a method of generating mutants, particularly sets of combinatorial mutants of the EphA agonistic peptides, as well as truncation mutants; pools of combinatorial mutants are especially useful for identifying functional variant sequences. The purpose of screening such combinatorial libraries may be to generate, for example, peptide variants which can act as either agonists or antagonist of EphA, or alternatively, which possess novel activities all together. A variety of screening assays are provided below, and such assays may be used to evaluate variants. For example, an EphA2 agonistic peptide variant may be screened for its ability to bind to an EphA2 polypeptide (full-length EphA2 or the LBD of EphA2) or for its ability to enhance binding of an EphA2 ligand to a cell expressing an EphA2 receptor. Optionally, an EphA agonistic peptide variant may be screened for its binding to EphA (full-length EphA or the LBD of EphA) with high affinity and specificity.

In other aspects, the EphA therapeutic agents of the present invention include a small molecule compound, such as those which activate EphA kinase function as well as those that suppress AKT kinase function and MAPK/ERK1/2 function. These small molecule compounds are also referred to herein as EphA agonistic compounds. Optionally, these agonistic compounds specifically target the ligand-binding domain of EphA kinase.

Exemplary, the EphA agonistic compounds include, but are not limited to compounds, such as dobutamine, labetalol, doxazosin, and (di-meo-isoquinolin-1-ylmethyl)-et-di-meo-pyrido(2,1-a)isoquinolin, hydrobromide S120103.

One aspect of the invention relates to a small molecule compound of Formula (I)

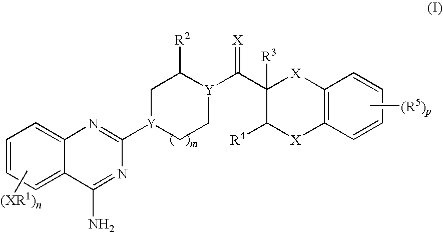

wherein
R¹ is selected from H, C₁₋₆alkyl, C₁₋₆aralkyl, C₁₋₆alkanoyl, aryl, heterocyclyl, C₁₋₆heterocyclylalkyl, C₁₋₆-carbocyclylalkyl, and carbocyclyl, or two occurrences of R¹ together are C₁₋₆alkyl, thereby forming a ring, preferably H or C₁₋₆alkyl;
R², R³, and R⁴ are independently selected from H, C₁₋₆alkyl, C₁₋₆aralkyl, C₁₋₆heterocyclylalkyl, and C₁₋₆carbocyclylalkyl, preferably R², R³, and R⁴ are independently selected from H and C₁₋₆alkyl;
R⁵ is selected from C₁₋₆alkyl, C₁₋₆alkoxy, halogen, C₁₋₆alkanoyl, C₁₋₆alkoxycarbonyl, C(O)N(R⁶)(R⁷), and SO₂N(R⁶)(R⁷);
R⁶ and R⁷ are independently selected from H and C₁₋₆alkyl;
X is selected from CH₂, NH, and O, preferably O;
each Y is independently selected from CH and N, preferably N;
m is an integer from 1 to 2, preferably 1;
n is an integer from 1 to 3, preferably 2; and
p is an integer from 0 to 3, preferably 0.

In certain embodiments, R¹ is selected from H and C₁₋₆alkyl, X is O, and n is an integer from 1 to 3. In preferred embodiments, n is 2, X is O, and R¹ is C₁₋₆alkyl.

In more preferred embodiments, n is 2, X is O, R¹ is methyl, and the substituents are located at the 3- and 4-positions of the aromatic ring.

In certain embodiments, R², R³, and R⁴ are independently selected H and C₁₋₆ lower alkyl. In preferred embodiments, R², R³, and R⁴ are all H.

In certain embodiments, m is 1 and each Y is independently selected from CH and N. In preferred embodiments, m is 1 and each occurrence of Y is N.

Another aspect of the invention relates to a compound comprising at least one aryl or heteroaryl ring. In preferred such embodiments, candidate compounds comprise two aryl or heteroaryl rings connected by a linker that is 5-10 atoms in length. In more preferred embodiments, candidate compounds have a structure of Formula (II)

A-L-B (II)

wherein
A and B are independently selected from aryl and heteroaryl; and
L is selected from C₁₋ᵢ₂alkyl and —C₁₋₆alkyl-amino-C₁₋₆alkyl-.

In certain embodiments, candidate compounds have a structure of Formula (III)

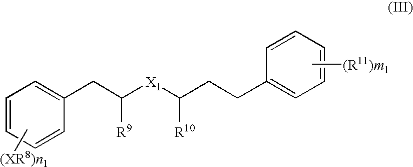

wherein
R⁸ is selected from H, C₁₋₆alkyl, C₁₋₆aralkyl, C₁₋₆alkanoyl, aryl, heterocyclyl, C₁₋₆heterocyclylalkyl, C₁₋₆carbocyclylalkyl, and carbocyclyl, or two occurrences of R¹ together are C₁₋₆alkyl, thereby forming a ring; R⁹ and R¹⁰ are independently selected from H, C₁₋₆alkyl, C₁₋₆aralkyl, C₁₋₆heterocyclylalkyl, and C₁₋₆-carbocyclylalkyl; R¹¹ is selected from H, OH, C₁₋₆alkoxy, and C₁₋₆alkanoyl, preferably OH; X₁ is selected from NH and O; m₁ is an integer from 1 to 2; and n₁ is an integer from 1 to 3.

In certain embodiments, R⁸ is selected from H and C₁₋₆alkyl, X is NH, and n is an integer from 1 to 3. In other embodiments, n is 2, X is NH, and R⁸ is H. In still other embodiments, n is 2, X is NH, R⁸ is H, and the substituents are located at the 3- and 4-positions of the aromatic ring.

In certain embodiments, R⁹ and R¹⁰ are independently selected from H and C₁₋₆alkyl. In other embodiments, R⁹ is H and R³ is C₁₋₆alkyl. In more preferred embodiments, R² is H and R³ is methyl.

In certain embodiments, m₁ is 1 and R¹¹ is selected from H, OH, C₁₋₆alkoxy, and C₁₋₆alkanoyl. In other embodiments, m₁ is 1 and R¹¹ is OH. In still other preferred embodiments, m₁ is 1, R¹¹ is OH, and the R¹¹ substituent is located at the 4-position of the aromatic ring.

As used herein, the term “C₁₋₆alkanoyl,” can be represented by the general formula:

—C(O)—C₁₋₆alkyl.

The term “C_{x-y}alkyl” refers to substituted or unsubstituted saturated hydrocarbon groups, including straight-chain alkyl and branched-chain alkyl groups that contain from x to y carbons in the chain, including haloalkyl groups, such as trifluoromethyl and 2,2,2-trifluoroethyl, etc. C0 alkyl indicates a hydrogen where the group is in a terminal position, a bond if internal.

The term “alkoxy” refers to an alkyl group having an oxygen attached thereto. Representative alkoxy groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxy.

The term “C₁₋₆alkoxycarbonyl”, as used herein can be represented by the general formula

—C(O)OC₁₋₆alkyl.

The term “C₁₋₆aralkyl”, as used herein, refers to a C₁₋₆alkyl group substituted with an aryl group.

The term “aryl” as used herein includes 5-, 6-, and 7-membered substituted or unsubstituted single-ring aromatic groups in which each atom of the ring is carbon. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The terms “carbocycle” and “carbocyclyl”, as used herein, refer to a non-aromatic substituted or unsubstituted ring in which each atom of the ring is carbon. The terms “carbocycle” and “carbocyclyl” also include polycyclic ring systems having two or more cyclic rings in which two or more carbons are common to two adjoining rings wherein at least one of the rings is carbocyclic, e.g., the other cyclic rings can be cycloalkyls, cycloalkenyls, cycloalkynyls, aryls, heteroaryls, and/or heterocyclyls.

The term “C₁₋₆-carbocyclylalkyl”, as used herein, refers to a C₁₋₆alkyl group substituted with a carbocycle.

The term “carbonyl” is art-recognized and includes such moieties as can be represented by the general formula:

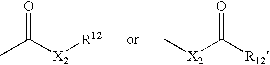

wherein X₂ is a bond or represents an oxygen or a sulfur, and R¹² represents a hydrogen, an alkyl, an alkenyl, —(CH₂)ₘ—R⁸ or a pharmaceutically acceptable salt, R^{12′} represents a hydrogen, an alkyl, an alkenyl or —(CH₂)ₘ—R⁸, where m and R⁸ are as defined above. Where X₂ is an oxygen and R¹² or R^{12′} is not hydrogen, the formula represents an “ester”. Where X₂ is an oxygen, and R¹² is a hydrogen, the formula represents a “carboxylic acid”.

The term “heteroatom” as used herein means an atom of any element other than carbon or hydrogen. Preferred heteroatoms are nitrogen, oxygen, phosphorus, and sulfur.

The terms “heterocyclyl” or “heterocyclic group” refer to substituted or unsubstituted non-aromatic 3- to 10-membered ring structures, more preferably 3- to 7-membered rings, whose ring structures include one to four heteroatoms. The term terms “heterocyclyl” or “heterocyclic group” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “C₁₋₆heterocycloalkyl” refers to a C₁₋₆alkyl group substituted with a heterocyclic group.

The terms “polycyclyl” or “polycyclic” refer to two or more rings (e.g., cycloalkyls, cycloalkenyls, cycloalkynyls, aryls, heteroaryls, and/or heterocyclyls) in which two or more carbons are common to two adjoining rings, e.g., the rings are “fused rings”. Each of the rings of the polycycle can be substituted or unsubstituted.

In certain aspects, the EphA therapeutic agents of the present invention include a peptidomimetic, for example, peptide-like molecules. As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of an EphA polypeptide (e.g., an EphA2 LBD).

As described herein, small molecule compounds may encompass numerous chemical classes, though typically they are organic molecules, preferably small organic compounds having a molecular weight of more than about 50 and less than about 2,500 daltons. Candidate agents comprise functional groups necessary for structural interaction with proteins, particularly hydrogen bonding, and typically include at least an amine, carbonyl, hydroxyl, sulfhydryl or carboxyl group. Candidate small molecule compounds can be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant, and animal extracts are available or readily produced. Additionally, natural or synthetically produced libraries and compounds can be modified through conventional chemical, physical, and biochemical means. Known pharmacological agents may be subjected to directed or random chemical modifications, such as acylation, alkylation, esterification, and amidification, to produce structural analogs.

In certain aspects, the EphA2 therapeutic agents include antibodies, for example, antibodies that are specifically reactive with an EphA2. Antibodies may be polyclonal or monoclonal; intact or truncated, e.g., F(ab′)2, Fab, Fv; xenogeneic, allogeneic, syngeneic, or modified forms thereof, e.g., humanized, chimeric, etc.

For example, by using immunogens derived from an EphA2 polypeptide, anti-protein/anti-peptide antisera or monoclonal antibodies can be made by standard protocols (see, for example, Antibodies: A Laboratory Manual ed. by Harlow and Lane (Cold Spring Harbor Press: 1988)). A mammal, such as a mouse, a hamster or rabbit can be immunized with an immunogenic form of the peptide. (e.g., a polypeptide or an antigenic fragment which is capable of eliciting an antibody response, or a fusion protein). Techniques for conferring immunogenicity on a protein or peptide include conjugation to carriers or other techniques well known in the art. An immunogenic portion of an EphA2 polypeptide can be administered in the presence of adjuvant. The progress of immunization can be monitored by detection of antibody titers in plasma or serum. Standard ELISA or other immunoassays can be used with the immunogen as antigen to assess the levels of antibodies. In one embodiment, antibodies of the invention are specific for the extracellular portion (e.g., the LBD) of the EphA protein (e.g., EphA1, EphA2, or EphA3). In another embodiment, antibodies of the invention are specific for the intracellular portion or the transmembrane portion of the EphA protein (e.g., EphA1, EphA2, or EphA3). In a further embodiment, antibodies of the invention are specific for the extracellular portion of the EphA2 protein (e.g., EphA1, EphA2, or EphA3).

Following immunization of an animal with an antigenic preparation of an EphA polypeptide (e.g., EphA1, EphA2, or EphA3), antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with an EphA2 polypeptide and monoclonal antibodies isolated from a culture comprising such hybridoma cells.

The term antibody as used herein is intended to include fragments thereof which are also specifically reactive with an EphA polypeptides. Antibodies can be fragmented using conventional techniques and the fragments screened for utility in the same manner as described above for whole antibodies. For example, F(ab)2 fragments can be generated by treating antibody with pepsin. The resulting F(ab)2 fragment can be treated to reduce disulfide bridges to produce Fab fragments. The antibody of the present invention is further intended to include bispecific, single-chain, and chimeric and humanized molecules having affinity for an EphA polypeptide conferred by at least one CDR region of the antibody. Techniques for the production of single chain antibodies (U.S. Pat. No. 4,946,778) can also be adapted to produce single chain antibodies. Also, transgenic mice or other organisms including other mammals, may be used to express humanized antibodies. In preferred embodiments, the antibodies further comprises a label attached thereto and able to be detected (e.g., the label can be a radioisotope, fluorescent compound, enzyme or enzyme co-factor).

In certain embodiments, an antibody of the invention is a monoclonal antibody, and in certain embodiments the invention makes available methods for generating novel antibodies. For example, a method for generating a monoclonal antibody that binds specifically to an EphA polypeptide (e.g., EphA1, EphA2, or EphA3) may comprise administering to a mouse an amount of an immunogenic composition comprising the EphA polypeptide (e.g., EphA1, EphA2, or EphA3) effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monoclonal antibody that binds specifically to the EphA polypeptide. Once obtained, a hybridoma can be propagated in a cell culture, optionally in culture conditions where the hybridoma-derived cells produce the monoclonal antibody that binds specifically to the EphA polypeptide. The monoclonal antibody may be purified from the cell culture.

In addition, the techniques used to screen antibodies in order to identify a desirable antibody may influence the properties of the antibody obtained. For example, an antibody to be used for certain therapeutic purposes will preferably be able to target a particular cell type. Accordingly, to obtain antibodies of this type, it may be desirable to screen for antibodies that bind to cells that express the antigen of interest (e.g., by fluorescence activated cell sorting). Likewise, if an antibody is to be used for binding an antigen in solution, it may be desirable to test solution binding. A variety of different techniques are available for testing antibody:antigen interactions to identify particularly desirable antibodies. Such techniques include ELISAs, surface plasmon resonance binding assays (e.g. the Biacore binding assay, Bia-core AB, Uppsala, Sweden), sandwich assays (e.g. the paramagnetic bead system of IGEN International, Inc., Gaithersburg, Md.), western blots, immunoprecipitation assays and immunohistochemistry.

The present invention also contemplates anti-tumor therapeutic agents obtainable from the screening methods described as below under “Methods of Screening.”

The present invention also provides methods of treating an individual suffering from cancer through administering to the individual a therapeutically effective amount of an EphA therapeutic agent as described above. The present invention provides methods of preventing or reducing the onset of cancer in an individual through administering to the individual an effective amount of an EphA therapeutic agent. These methods are particularly aimed at therapeutic and prophylactic treatments of animals, and more particularly, humans.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

Cancers and related disorders that can be treated, prevented, or managed by methods, EphA therapeutic agents and compositions of the present invention include but are not limited to cancers include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America)

Accordingly, the methods and EphA therapeutic agents of the invention are also useful in the treatment or prevention of a variety of cancers or other abnormal proliferative diseases, including (but not limited to) the following: carcinoma, including that of the bladder, breast, prostate, rectal, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyclocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the skin, lung, colon, rectum, breast, prostate, bladder, kidney, pancreas, ovary, or uterus. In other specific embodiments, sarcoma, melanoma, or leukemia is treated or prevented.

In some embodiments, the cancer is malignant and overexpresses EphA. In other embodiments, the disorder to be treated is a pre-cancerous condition associated with cells that overexpress EphA. In a specific embodiments, the pre-cancerous condition is high-grade prostatic intraepithelial neoplasia (PIN), fibroadenoma of the breast, fibrocystic disease, or compound nevi.

In certain embodiments, EphA therapeutic agents of the invention can be delivered to cancer cells by site-specific means. Cell-type-specific delivery can be provided by conjugating a therapeutic agent to a targeting molecule, for example, one that selectively binds to the affected cells. Methods for targeting include conjugates, such as those described in U.S. Pat. No. 5,391,723. Targeting vehicles, such as liposomes, can be used to deliver a compound, for example, by encapsulating the compound in a liposome containing a cell-specific targeting molecule. Methods for targeted delivery of compounds to particular cell types are well-known to those skilled in the art.

In certain embodiments, one or more EphA therapeutic agents can be administered, together (simultaneously) or at different times (sequentially). In addition, therapy by administration of one or more EphA therapeutic agents can be combined with the administration of one or more therapies such as, but not limited to, chemotherapies, radiation therapies, hormonal therapies, and/or biological therapies/immunotherapies. Prophylactic/therapeutic agents include, but are not limited to, proteinaceous molecules, including, but not limited to, peptides, polypeptides, proteins, including post-translationally modified proteins, antibodies etc.; or small molecules (less than 1000 daltons), inorganic or organic compounds; or nucleic acid molecules including, but not limited to, double-stranded or single-stranded DNA, or double-stranded or single-stranded RNA, as well as triple helix nucleic acid molecules. Prophylactic/therapeutic agents can be derived from any known organism (including, but not limited to, animals, plants, bacteria, fungi, and protista, or viruses) or from a library of synthetic molecules.

In one embodiment the EphA therapeutic agents can be administered in conjunction with an immunosuppressant, such as a mammalian target of rapamycin (mTOR) inhibitor to treat proliferative or neoplastic disorders (e.g., cancer or tumors), which are associated EphA kinase expression or over expression by the neoplastic cells (e.g., tumor or cancer cells). The phosphatidylinositol 3-kinase (PI3-K)/Akt/mammalian target of rapamycin (mTOR) and the Ras/extracellular signal-regulated kinase 1/2 (ERK1/2) mitogenic signaling pathways have been shown to contribute to the development and progression of neoplastic disease as well as therapeutic resistance. The PI3-K/Akt/mTOR pathway is activated in about 20% to about 50% of prostate cancers predominately due to the mutation or inactivation of the tumor suppressor PTEN. Akt is an oncoprotein that has been identified as a key downstream effector of the PI3-K signaling pathway. The binding of PI3-K-generated phospholipids to Akt results in the translocation of Akt from cytoplasm to the inner surface of the membrane, where Akt is then fully activated by phosphorylation on Thr³⁰⁸ and Ser⁴⁷³. Active Akt in human cancers are believed to promote cell survival, proliferation, motility, and invasion, thereby contributing to tumor progression. Increased Akt phosphorylation is detected in prostate cancers with a high Gleason grade and is an excellent predictor of poor clinical outcome. One of the most relevant mediators of Akt functions is mTOR, which is known to regulate cell cycle progression and cell growth.

Similarly, the RAS/ERK1/2 pathway is primarily known for mitogenic signaling and for modulating cell survival in most model systems. ERK1/2 is constitutively expressed and activated in a significant proportion of human prostate cancer cell lines as well as tumor tissues, and likely play a causal role in the progression of this malignancy from adrogen-sensitive to adrogen-insensitive metastatic disease.

In light of the evidence for the crucial roles of both pathways in cancer development and progression, an aspect of the invention includes a method of inhibiting both Akt and ERK1/2 kinase activity in a neoplastic cell by administering an EphA therapeutic agent and an immunosupressant, such as an mTOR inhibitor, to the neoplastic cell expressing EphA kinase. Still another aspect of the invention relates to a method of treating neoplastic disease in a subject including administering an EphA agonist to a subject in combination with administering one or more immunosuppressants. The method in accordance with the present invention can comprise the administration of the EphA agonist to modulate an EphA kinase in a cell of the subject. Modulating an EphA kinase can include activating and overexpressing EphA kinase in a cell of the subject.

The immunosuppressant in accordance with the present invention can include a mammalian target of rapamycin (mTOR) inhibitor. An mTOR inhibitor can be any mTOR inhibitor that is currently known in the art or that will be identified in the future, and includes any chemical entity that, upon administration to a patient, results in inhibition of mTOR in the patient. An mTOR inhibitor can inhibit mTOR by any biochemical mechanism, including competition at the ATP binding site, competition elsewhere at the catalytic site of mTOR kinase, non-competitive inhibition, irreversible inhibition (e.g., covalent protein modification), or modulation of the interactions of other protein subunits or binding proteins with mTOR kinase in a way that results in inhibition of mTOR kinase activity (e.g., modulation of the interaction of mTOR with FKBP12, GβL, (mLST8), RAPTOR (mKOG1), or RICTOR (mAVO3)). Specific examples of mTOR inhibitors include: rapamycin (e.g., sirolimus); other rapamycin macrolides, or rapamycin analogues, derivatives or prodrugs; RAD001 (also known as Everolimus, RAD001 is an alkylated rapamycin (40-O-(2-hydroxyethyl)-rapamycin), disclosed in U.S. Pat. No. 5,665,772; Novartis); CCI-779 (also known as Temsirolimus, CCI-779 is an ester of rapamycin (42-ester with 3-hydroxy-2-hydroxymethyl-2-methylpropionic acid), disclosed in U.S. Pat. No. 5,362,718; Wyeth); AP23573 or AP23841 (Ariad Pharmaceuticals); ABT-578 (40-epi-(tetrazolyl)-rapamycin; Abbott Laboratories); KU-0059475 (Kudus Pharmaceuticals); and TAFA-93 (a rapamycin prodrug; Isotechnika).

Examples of rapamycin analogs and derivatives known in the art include those compounds described in U.S. Pat. Nos. 6,329,386; 6,200,985; 6,117,863; 6,015,815; 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; and 5,023,262; all of which are incorporated herein by reference.

Rapamycin derivatives are also disclosed for example in WO 94/09010, WO 95/16691, WO 96/41807, or WO 99/15530, which are incorporated herein by reference. Such analogs and derivatives include 32-deoxorapamycin, 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-O-(2-hydroxyethyl)-rapamycin, 40-O-(2-hydroxyethyl)-rapamycin, 32-deoxorapamycin and 16-pent-2-ynyloxy-32(S)-dihydro-rapamycin. Rapamycin derivatives may also include the so-called rapalogs, e.g., as disclosed in WO 98/02441 and WO01/14387 (e.g. AP23573, AP23464, AP23675 or AP23841). Further examples of a rapamycin derivative are those disclosed under the name biolimus-7 or biolimus-9 (BIOLIMUS A9™) (Biosensors International, Singapore). Any of the above rapamycin analogs or derivatives may be readily prepared by procedures as described in the above references.

Additional examples of mTOR inhibitors useful in the invention described herein include those disclosed and claimed in U.S. patent application Ser. No. 11/599,663, filed Nov. 15, 2006, a series of compounds that inhibit mTOR by binding to and directly inhibiting both mTORC1 and mTORC2 kinases.

Also useful in the invention described herein are mTOR inhibitors that are dual PI3K/mTOR kinase inhibitors, such as for example the compound PI-103 as described in Fan, Q-W et al (2006) Cancer Cell 9:341-349 and Knight, Z. A. et al. (2006) Cell 125:733-747.

The present invention also encompasses the use of a combination of an EphA therapeutic agent (e.g., EphA agonist) and an mTOR inhibitor, for the manufacture of a medicament for the treatment of lung, breast, prostate, brain, or colon cancer or tumors or other cancers or tumor metastases in which an EphA kinase is expressed in cancer or tumor cell and wherein each agent in the combination can be administered to the patient either simultaneously or sequentially. The present invention also encompasses the use of a synergistically effective combination of an EphA agonist and an mTOR inhibitor, for the manufacture of a medicament for the treatment of lung, breast, prostate, brain, or colon cancer or tumors or other cancers or tumor metastases in which EphA kinase is expressed in cancer or tumor cell and wherein each agent in the combination can be administered to the patient either simultaneously or sequentially.

The invention also encompasses a pharmaceutical composition that is comprised of a combination of an EphA therapeutic agent and an mTOR inhibitor in combination with a pharmaceutically acceptable carrier.

The pharmaceutical composition can include a pharmaceutically acceptable carrier and a non-toxic therapeutically effective amount of a combination of an EphA therapeutic agent and an mTOR inhibitor (including pharmaceutically acceptable salts of each component thereof).

Moreover, the invention encompasses a pharmaceutical composition for the treatment of disease, the use of which results in the inhibition of growth of neoplastic cells, benign or malignant tumors, or metastases, comprising a pharmaceutically acceptable carrier and a non-toxic therapeutically effective amount of a combination of an EphA therapeutic agent and an mTOR inhibitor (including pharmaceutically acceptable salts of each component thereof).

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When a compound of the present invention is basic, its corresponding salt can be conveniently prepared from pharmaceutically acceptable non-toxic acids, including inorganic and organic acids. Such: acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The pharmaceutical compositions of the present invention comprise a combination an EphA therapeutic agent and an mTOR inhibitor (including pharmaceutically acceptable salts of each component thereof) as active ingredients, a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by the combination of an EphA therapeutic agent and an mTOR inhibitor (including pharmaceutically acceptable salts of each component thereof) of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, a combination of an EphA therapeutic agent and an mTOR inhibitor (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a combination of an EphA therapeutic agent and an mTOR inhibitor (including pharmaceutically acceptable salts of each component thereof). A combination of an EphA therapeutic agent and an mTOR inhibitor (including pharmaceutically acceptable salts of each component thereof), can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above.

Thus in one embodiment of this invention, a pharmaceutical composition can comprise a combination of an EphA therapeutic agent and an mTOR inhibitor in combination with an anticancer agent, wherein said anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical sue such as, for example, an aerosol, cream, ointment, lotion, dusting powder, or the like. Further, the compositions can be in a form suitable for use in transdermal devices.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient.

Dosage levels for the compounds of the combination of this invention will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

The present invention also provides a method for treating tumors or tumor metastases in a patient, comprising administering to said patient simultaneously or sequentially a therapeutically effective amount of a combination of an EphA therapeutic agent and an mTOR inhibitor. In one embodiment of this method the patient is a human that is being treated for cancer. In one embodiment of this method the cells of the tumors or tumor metastases are relatively insensitive or refractory to treatment with an mTOR inhibitor as a single agent. In one embodiment of this method the EphA therapeutic agent (e.g., EphA2 agonist) and mTOR inhibitor are co-administered to the patient in the same formulation. In another embodiment of this method the EphA therapeutic agent (e.g., EphA2 agonist) and mTOR inhibitor are co-administered to the patient in different formulations. In another embodiment of this method the EphA therapeutic agent (e.g., EphA2 agonist) and mTOR inhibitor are co-administered to the patient by the same route. In another embodiment of this method the EphA therapeutic agent (e.g., EphA2 agonist) and mTOR inhibitor are co-administered to the patient by different routes.

The present invention also provides a method for treating tumors or tumor metastases in a patient, comprising the steps of diagnosing a patient's likely responsiveness to an EphA agonist by assessing whether the tumor cells express an EphA receptor (e.g., EphA2) and thus likely to show an enhanced response to treatment with an Epha agonist and an mTOR inhibitor, and administering to said patient simultaneously or sequentially a therapeutically effective amount of a combination of the EphA therapeutic agent (e.g., EphA2 agonist) and mTOR inhibitor.

### EXAMPLES

The invention now being generally described, it will be more readily understood by reference to the following examples which are included merely for purposes of illustration of certain aspects and embodiments of the present invention, and are not intended to limit the invention.

### Example 1

### Activation of EphA2 Kinase Inhibits Akt and ERK1/2 Activities in Prostate Cancer Cells and Potentiates Growth Inhibition by Rapamycin

### Materials and Methods

### Reagents and Antibodies

All chemicals were purchased from Fisher Scientific Co. (Pittsburgh, Pa.) unless otherwise indicated. Ephrin-A1-Fc and Fc were prepared as described previously. Fetal bovine serum (FBS) and RPMI 1640 were obtained from Hyclone (Logan, Utah) and Lerner Research Institute (Cleveland, Ohio), respectively. Mouse monoclonal anti-a-tubulin and 5-bromo-4-chloro-β-D-galactopyranoside (X-gal) were purchased from Sigma Chemical Co. (St Louis, Mo.). Rapamycin and puromycin were obtained from LC Laboratories (Woburn, Mass.) and Invivogen (San Diego, Calif.), respectively. Stock solution of rapamycin was prepared in dimethyl sulfoxide (DMSO) and stored at −20° C. Okadaic acid was obtained from Calbiochem (San Diego, Calif.). FuGENE HD Transfection Reagent, In Situ Cell Death Detection Kit, and protease inhibitor cocktail were purchased from Roche Applied Science (Indianapolis, Ind.). Bio-Rad Dc Protein Assay kit and growth factor-reduced Matrigel were obtained from Bio-Rad Laboratories (Hercules, Calif.) and BD Biosciences (San Jose, Calif.), respectively. Rabbit polyclonal anti-phospho-ERK1/2 (p-ERK1/2), anti-total ERK1/2, goat polyclonal anti-PTEN, horseradish peroxidase-conjugated secondary antibodies, and Western Blotting Luminol Reagent were obtained from Santa Cruz Biotechnology (Santa Cruz, Calif.). Rabbit polyclonal anti-phospho-Akt (p-Akt; Ser473), anti-total Akt, anti-PI3-K p110a, and anti-PI3-K p85 were obtained from Cell Signaling Technology (Danvers, Mass.). Rabbit polyclonal anti-Ki-67 antibody and Texas red-conjugated secondary antibody were purchased from Novocastra Lab Ltd. (Newcastle, UK) and Jackson ImmunoResearch Laboratories (West Grove, Pa.), respectively.

### Cell Culture and Retroviral Infection

The parental DU145 cells, derived from a brain metastasis, were obtained from American Type Culture Collection (Manassas, Va.), and maintained in RPMI 1640 supplemented with 10% FBS, 29 mg/ml glutamine, 100 U/ml penicillin, and 100 μg/ml streptomycin at 37° C. in a humidified atmosphere containing 5% CO2. Cells at 70-80% confluence onto 6-well culture plates were stably infected with pBMC-puromycin bicistronic retroviral vector expressing wild-type EphA2 or empty vector to generate DU145-EphA2 and DU145-vector cells, respectively, followed by selection in 1 μg/ml puromycin. Expression of EphA2 was verified by immunoblot with a specific antibody.

Cell Treatment and Immunoblot Cells cultured in 6-well plates at 70-80% confluence were incubated with 2 μg/ml ephrin-A1-Fc alone or in combination with various drugs for the indicated times. Fc or DMSO (vehicle) was used for corresponding control. For serum/growth factor withdrawal experiments, cells were washed once with phosphate buffered saline (PBS, pH 7.4) and incubated with RPMI 1640 containing 0.5% FBS for 24 h. After treatment, cells were lysed in RIPA buffer (20 mM Tris, pH 7.4, 125 mM NaCl, 20 mM NaF, 0.1% SDS, 10% glycerol, 0.5% sodium deoxycholate, 1% Triton X-100, and 0.5 mM Na3VO4) containing protease inhibitors (2 μg/ml each of aprotinin and leupeptin, and 1 mM phenylmethylsulphonyl fluoride). Cell lysates were clarified by centrifugation at 13,000 rpm for 5 min, separated on 4-20% Tris-glycine gels (Invitrogen, Carlsbad, Calif.), and then transferred to Immobilon-P polyvinylidene fluoride (PVDF) membranes (Millipore, Bedford, Mass.). Membranes were blotted according to manufacturer's instructions.

### Colony Formation Assay

To test the effect of overexpression of wild-type EphA2 on the colony formation in DU145 cells, transient transfections were performed using FuGENE HD Transfection Reagent according to the manufacturer's instructions. Briefly, cells were plated onto 6-well plates in duplicate and co-transfected with 1 μg of β-galactosidase-expression plasmid and 5 μg of the plasmids encoding wild-type EphA2 or empty vector. After overnight incubation, duplicate plates of transfected cells were used for X-gal staining to gauge the transfection efficiency and colony formation assays. X-gal staining was performed, and the average number of β-galactosidase positive cells was determined by counting the stained blue cells under a microscope. Meanwhile, cells on the duplicate plates were passaged onto 35-mm dishes and grown in RPMI 1640 containing 1 μg/ml puromycin for 2 weeks. Then, cells were fixed with 4% paraformaldehyde and stained with 0.5% crystal violet to measure the colony formation. Colonies (>1 mm) were visually scored independently by two different researchers.

### Clonal Growth Assays In Vitro

Cells were seeded in 12-well plates in normal growth medium at a density of 2×103 cells per well in duplicate. The following day, cells were treated with ephrin-A1-Fc, rapamycin alone or in combination at the indicated concentrations. Fc or DMSO (vehicle) was used for corresponding control. Cells were incubated for 14 days, during which time the medium containing various indicated drugs was changed every 5 days. Cells were then stained with 0.5% crystal violet solution in 20% methanol. For proliferation assays using cell counting, 5×103 cells/well were seeded onto 6-well dishes and grown overnight before treatment with various indicated drugs. Cells were collected with trypsin-EDTA solution every 24 h, stained with Trypan blue, and counted using a hemocytometer. Each experiment was repeated three times.

### Xenograft Tumors in Athymic Mice

Male NCR-nu/nu athymic mice, 4-6 weeks old, were obtained from Case Comprehensive Cancer Center, Case Western Reserve University (Cleveland, Ohio), and housed in laminar-flow cabinets under specific pathogen-free conditions with food and water ad libitum. All experiments on mice were conducted in accordance with the guidelines of National Institutes of Health for the Care and Use of Laboratory Animals and Case Institutional Animal Care and Use Committee. For xenograft tumor assays, a total of 2×106 cells in 100 μl of PBS were mixed 1:1 with Matrigel and then inoculated subcutaneously into the dorsal flanks of mice. Each group consisted of six mice and two inoculations per mouse were performed. Primary tumor outgrowth was monitored twice weekly by taking measurements of the tumor length (L) and width (W). Tumor volume was calculated as pLW2/6. Mice were sacrificed at 8 weeks of post-inoculation, and the tumors were harvested for further analysis. The harvested tumors were either fixed in 10% formalin for hematoxylin and eosin (H&E) staining to assess cell morphology, frozen in Tissue Freezing Medium (Triangle Biomedical Sciences, Durham, N.C.) for indirect immunofluorescent (IF) staining, or extracted with lysis buffer for immunoblot analysis.

Expression of EphA2, p-Akt, and p-ERK1/2 in the Xenografted Tumors

The minced tumor pieces were homogenized in ice-cold RIPA lysis buffer supplemented with protease inhibitor cocktails. Lysates were cleared by centrifugation at 14,000 rpm for 10 min, and protein concentrations were determined by Bio-Rad Dc Protein Assay. Equal amounts of protein extracts were resolved by sodium dodecyl sulfate-polyacrylamide gel electrophoresis (SDS-PAGE) and electrotransferred onto PDVF membranes. Membranes were then blotted with antibodies against EphA2, p-Akt, and p-ERK1/2. The membranes were stripped and reprobed with antibodies that recognize total Akt, ERK1/2, or a-tubulin to confirm similar sample loading.

### Proliferative and Apoptotic Assays

Cell proliferation was assessed using indirect IF staining. Briefly, air-dry frozen tissue sections were fixed with 4% paraformaldehyde for 20 min, blocked with 50 mM NH4Cl, and permeabilized with 0.3% NP-40 for 10 min. Then, cells were incubated with rabbit polyclonal anti-Ki-67 (1:1000) at RT for 1 h, followed by detection with Texas red-conjugated donkey anti-rabbit secondary antibody (1:300) at RT for 30 min. Vectashield mounting medium containing 4′-6-diamidino-2-phenylindole (DAPI) (Vector Laboratories, Burlingame, Calif.) was used to mount the coverslips onto slides. Images were taken using a Leica microscope with MetaMorph 6.2v4 imaging software (Universal Imaging, Downingtown, Pa.). Non-specific background staining were controlled for by using secondary antibody with pre-immune primary IgG. The proliferative index was determined as the number of Ki67-stained nuclei/total number of DAPI-stained nuclei in 10 randomly selected fields at 20× magnification. Analysis of apoptotic cells was performed by using terminal deoxynucleotidyl transferase-mediated UTP end labeling (TUNEL) staining kit according to the manufacturer's instructions. Negative controls were done by omission of enzyme solution from the TUNEL reaction mixture. TUNEL-positive nuclei were counted in 10 randomly selected fields at 20× magnification, and compared with DAPI-stained nuclei to determine the percentage of apoptosis.

### Statistical Analysis

Data are expressed as the mean±SD of three independent experiments. Graphs were created using MicroCal Origin 6.0 software (OriginLab Corporation, Northampton, Mass.). Statistical analysis was performed using the software of Statistical Package for the Social Sciences (SPSS) version 11.5 for Windows (Chicago, Ill.), and P<0.05 was considered as statistically significant.

### Results

Kinase Activation of EphA2 in DU145 Cells Simultaneously Inhibits both Akt and ERK1/2 Activities

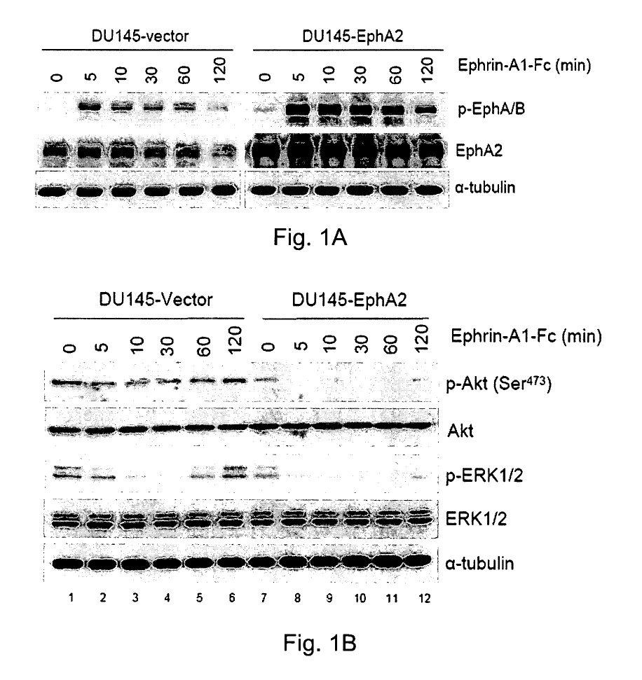

To simulate EphA2 overexpression in human cancers and to directly test the functional role of EphA2 kinase in human prostate cancer, we overexpressed EphA2 in DU145 prostate cancer cells by retroviral transduction. Stable clones were established that overexpressed EphA2 by 3-4 folds over control retroviral vector-transduced cells (DU145-vector). To study the biochemical consequences of ligand stimulation of EphA2, cells were treated with 2 μg/ml ephrin-A1 fused to human IgG1 heavy chain (ephrin-A1-Fc) for different times. Immunoblot analyses showed that ephrin-A1 stimulation led to a rapid activation of EphA2 as detected by an antibody raised against the phosphorylated di-tyrosine motif conserved among all Eph kinases (FIG. 1A).

PI3-K/Akt and Ras/ERK1/2 pathways are major players of the mitogenic and antiapoptotic response in prostate cancer cells. To investigate the role of EphA2 in regulating both pathways, DU145-EphA2 and vector control cells were treated with ephrin-A1 and analyzed for activation status of Akt and ERK1/2 kinases. FIG. 1B shows that activation of endogenous EphA2 not only inhibited the activities of ERK1/2 kinase, but also significantly attenuated Akt activation status as indicated by the p-Akt (Ser473) signal. Forced expression of EphA2 in DU145 cells led to a marked decrease in basal levels of p-Akt and p-ERK1/2 compared with vector control (FIG. 1 B; compare lanes 1 and 7); the inhibitory effects were enhanced by ligand stimulation (FIG. 1 B). Notably, while stimulation of DU145-vector cells with ephrin-A1 caused a transient decrease in Akt and ERK1/2 phosphorylation that recovered to basal levels by 2 h after ligand stimulation, the dephosphorylation of Akt and ERK1/2 in EphA2-overexpressing cells was much more pronounced and lasted for longer duration. Collectively, these results suggest that activation of endogenous EphA2 in DU145 cells can simultaneously suppress both Akt and ERK1/2 activities, which is further augmented by EphA2 overexpression. Similar results were obtained from PC3 prostate cancer cells, and a subset of lung and breast cancer cell lines (not shown).

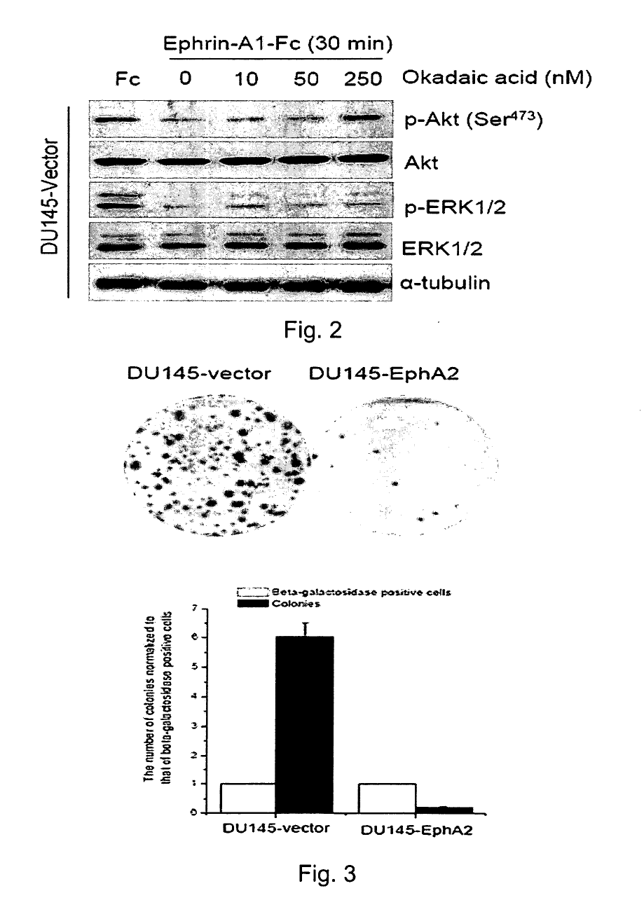

We next tested whether EphA2 overexpression and activation modulated by upstream regulators of Akt, PTEN and PI3-K expression, We found that forced expression and activation of EphA2 did not affect PTEN and PI3-K expression in DU145 cells (data not shown). Akt activity is also regulated by Ser/Thr phosphatases, such as phosphatase 2A (PP2A) or protein phosphatase 1 (PP1). To determine whether dephosphorylation of Akt by EphA2 activation is mediated through activation of phosphatase, we pretreated DU145-vector cells for 1 h with okadaic acid, a potent PP2A and PP1 inhibitor, followed by stimulation with ephrin-A1 for 30 min. As shown in FIG. 2, okadaic acid did not affect Akt dephosphorylation induced by EphA2 activation at 10 and 50 nM but completely blocked p-Akt at 250 nM. The effects on ERK1/2 under the same conditions were not as pronounced, suggesting that different mechanisms may be involved in EphA2-mediated regulation of ERK1/2 and Akt. Similar results were also obtained from a non-small cell lung cancer cell line expressing high level of endogenous EphA2 (data not shown). Previous studies have revealed that okadaic acid inhibits only PP2A at low concentrations (<100 nM), but it inhibits both PP1 and PP2A at higher concentrations (>100 nM). These results suggest EphA2 activation promotes Akt dephosphorylation by activating PP1 rather than PP2A.

### Overexpression of EphA2 Inhibits Cell Growth and Sensitizes Cells to Serum Withdrawal

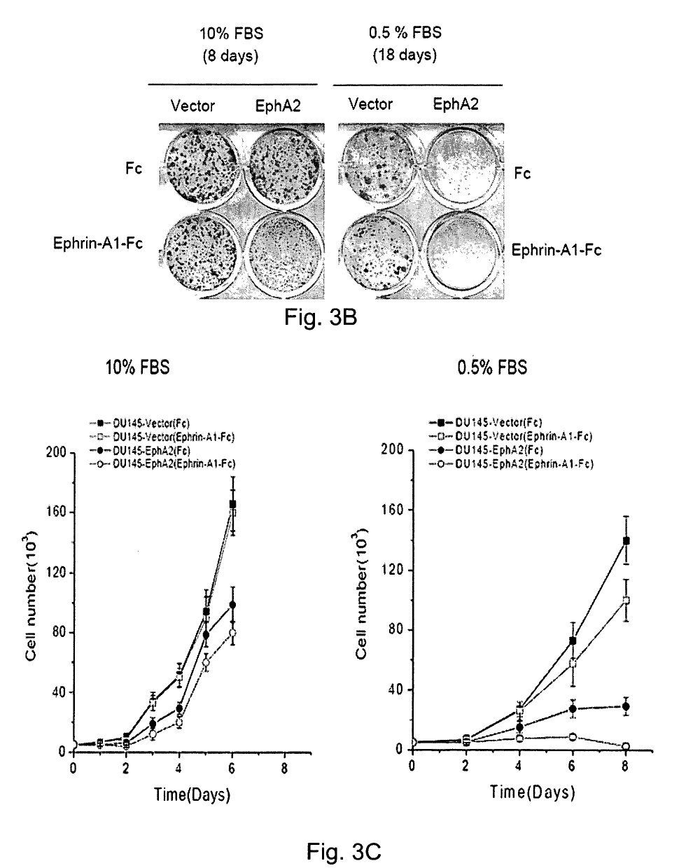

Numerous studies have demonstrated that growth factor signaling through the PI3-K/Akt and Ras/ERK1/2 pathways promotes cell survival and proliferation. Given that forced expression and activation of EphA2 in DU145 cells markedly suppressed Akt and ERK1/2 activities, we next examined whether EphA2 overexpression affected cell growth in vitro. For this purpose, we first transiently co-transfected DU145 cells with a β-galactosidase-expression plasmid together with an EphA2-expression plasmid or vector control. One set of plates was stained 48 h later with X-gal to gauge transfection efficiency; another set of plates was assayed for colony formation in the presence of puromycin selection marker. DU145 cells over-expressing EphA2 showed dramatically suppressed colony formation relative to control cells (FIG. 3A). To further confirm the results, clonal growth assays were performed using stably infected DU145 cells. Equal numbers of DU145-EphA2 and DU145-vector cells (2×103 cells/well) seeded on 12-well cluster plates were allowed to grow in the presence of ephrin-A1-Fc or Fc control for indicated times. Consistent with colony formation assay above, when cultured in 10% serum, DU145-EphA2 cells grew slower than DU145-vector cells. Addition of ephrin-A1-Fc significantly inhibits the clonal growth of DU145-EphA2 cells but not vector control cells (FIG. 3B; left panel). Interestingly, if the serum concentration was reduced to 0.5%, DU145-EphA2 cells grew much slower than vector control cells, and were largely growth-arrested in the presence of ephrin-A1 (FIG. 3B; right panel).

We next validated the dramatic effects of serum withdrawal on clonal growth of EphA2 overexpressing cells by monitoring daily changes in total cell numbers. Consistent with clonal growth assay, EphA2-overexpressing cells showed enhanced sensitivity to serum withdrawal relative to controls (FIG. 3C; compare left and right panels), which was further augmented by ephrin-A1-Fc treatment. Indeed, the combination of low serum and ephrin-A1 caused full growth arrest of DU145-EphA2 cells. In contrast, growth of DU145-vector cells was largely unaffected by ephrin-A1 either in normal or serum-deprived conditions. Together these data demonstrate that EphA2 overexpression dramatically sensitized cells to growth inhibition induced by serum withdrawal, which was further accentuated by ephrin-A1 treatment.

To determine the molecular basis for these growth inhibitory effects, cells were cultured in the medium containing 0.5% serum for 24 h and then stimulated with ephrin-A1-Fc for 10 and 30 min. Immunoblot analysis showed that overexpression of EphA2 in DU145-EphA2 cells decreased the basal levels of p-Akt (Ser473) and p-ERK1/2 relative to control cells, and the inhibitory effects were further enhanced by ligand stimulation (FIG. 3D). These results suggest that Akt- and ERK1/2-mediated cell growth signaling is reduced by overexpression of EphA2.

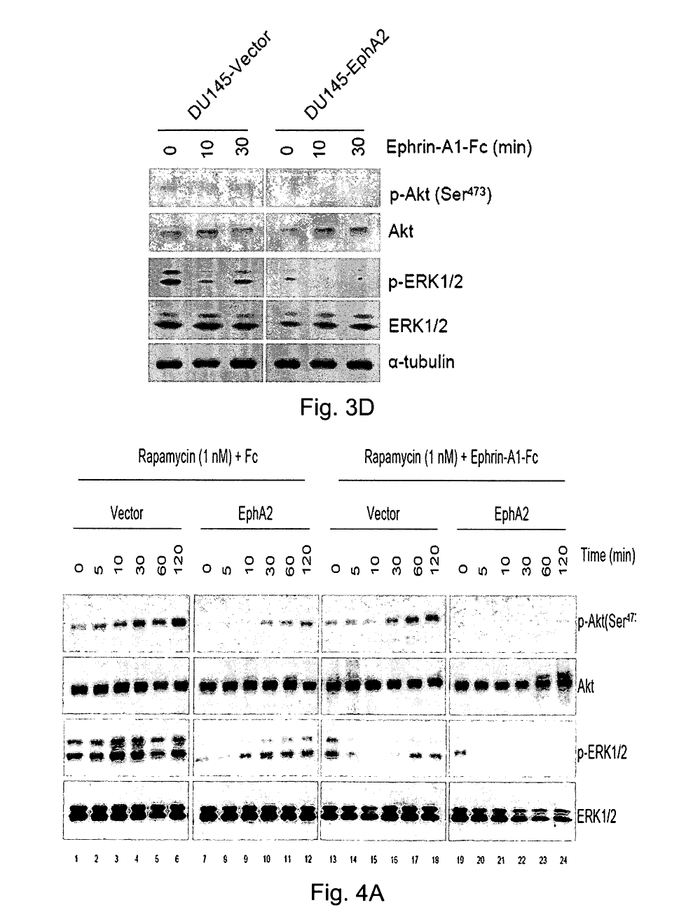

Overexpression of EphA2 sensitizes DU145 cells to the growth inhibition by rapamycin Recently, the compensatory feedback activation of Akt by rapamycin has been observed in several model systems which might attenuate antitumor effects of rapamycin. Accordingly, abrogating this induction might enhance its antitumor effects. Because overexpression of EphA2 in DU145 cells led to a marked decrease in p-Akt, we were interested in finding out whether EphA2 activation could suppress Akt induction by rapamycin. For this purpose, DU145-vector and DU145-EphA2 cells were treated with rapamycin in the absence or presence of ephrin-A1 for different times. As shown in FIG. 4A (lanes 1-6), treatment of DU145-vector cells with 1 nM rapamycin increased the phosphorylation of Akt in a time-dependent manner. Interestingly, we found here for the first time that ERK1/2 activities were also upregulated by rapamycin following similar kinetics. EphA2-overexpressing cells showed significantly lower basal levels of Akt and ERK1/2 activities compared with vector control cells (FIG. 4A; compare lane 1 and 7). Rapamycin was still able to induce both Akt and ERK1/2 activation, although the overall levels of activities were substantially lower than in vector cells (FIG. 4A lanes 7-12). Upon ephrin-A1 stimulation, rapamycin induction of Akt and ERK1/2 activities was significantly reduced in vector cells (FIG. 4A lanes 13-18), and was nearly abolished in EphA2-overexpressing cells (FIG. 4A lanes 19-24). Next, we tested whether overexpression of EphA2 could enhance the anti-proliferative effects of rapamycin using clonal growth assays. FIG. 4B shows that overexpression of EphA2 sensitized DU145 cells to growth inhibition induced by rapamycin or ephrin-A1 compared with vector control cells. Moreover, a cooperative effect of growth inhibition was observed when rapamycin was added in combination with ephrin-A1. These results were further confirmed by cell counting in a separate experiment (FIG. 4C). The growth curves showed that the proliferation of vector control cells expressing low levels of EphA2 was minimally affected by ephrin-A1 but was inhibited by rapamycin in a dose-dependent manner. EphA2 overexpression caused a significantly increased responsiveness to ephrin-A1 from day 2 after the initiation of treatment and persisted for the entire duration of the experiment. Moreover, co-treatment with ephrin-A1 and rapamycin had cooperative inhibitory effects; at 100 nM rapamycin there was a near complete suppression of cell proliferation. Therefore, EphA2 overexpression sensitized cells to growth inhibition by ephrin-A1; rapamycin co-treatment has a cooperative effect. Collectively, these results suggest that overexpression and activation of EphA2 receptor sensitize DU145 cells to growth inhibition induced by rapamycin in vitro through, at least in part, disruption of the Akt and ERK1/2 feedback loops.

### Overexpression of EphA2 Inhibits the Growth of DU145 Cells In Vivo

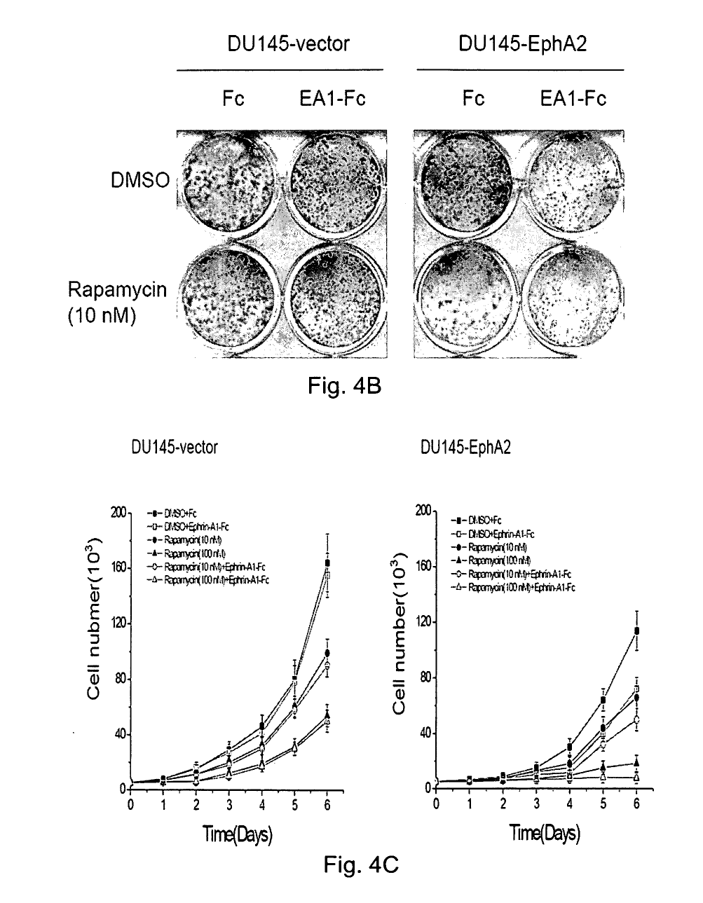

The significant suppression of basal Akt and ERK1/2 activities by EphA2 overexpression in vitro led us to assess the functional significance of EphA2 overexpression in the growth of prostate cancer cells in vivo. DU145 cells expressing wild-type EphA2 or empty vector were injected into both flanks of athymic mice, and tumor volumes were monitored twice weekly. As shown in FIG. 5A, tumors expressing wild-type EphA2 showed a slower tumor growth rate than control tumors. Representative histological analysis of xenograft tumors is presented in FIG. 5B. Immunoblot analysis confirmed that EphA2 overexpression was retained in DU145-EphA2 tumors (FIG. 5C). In addition, the p-Akt activities were significantly lower in EphA2-overexpressing tumors than vector controls (FIG. 5C). A trend of lower ERK1/2 activities was also observed, although the difference is not statistically significant in a quantitative analysis. To assess the effect of overexpression of EphA2 on cell proliferation and apoptosis in vivo, we determined proliferation index by anti-Ki67 staining and apoptosis by TUNEL staining. FIG. 5D shows that Ki67-positive cells was significantly reduced in DU145-EphA2 tumors relative to vector control tumors (P=0.00139). In contrast, there was no significant difference in apoptosis between vector control and EphA2-tumors (P>0.05) (data not shown).

### Discussion

We reported here that forced expression and activation of EphA2 simultaneously inhibited Akt and ERK1/2 kinase activities. EphA2 overexpression inhibited cell growth, and markedly sensitized cells to growth factor withdrawal. Moreover, we provide evidence that enhanced expression of EphA2 promotes increased sensitivity cells to growth inhibition by rapamycin, indicating that levels of EphA2 may be an important determinant of the therapeutic efficacies of mTOR inhibitors. These results support our model that EphA2 kinase functions as a tumor suppressor in prostate cancer development, at least in part, through the inactivation of Akt and ERK1/2 pathways.

Abnormal expression of Eph receptors and/or their ligands has been documented in various human cancers. More recently, Eph kinases are found to be mutated in common solid tumors in humans. Mutational analysis of tyrosine kinome revealed somatic mutations in the kinase domain of EphA3 in human colorectal cancer. Using nonsense-mediated decay microarray analysis, frequent mutations (8%) of EphB2 kinase in human prostate cancer both in the ectodomain and the cytoplasmic tail have been reported, several of which are predicted to cause loss of function. Moreover, re-expression of wild-type EphB2 kinase in DU145 cells can inhibit their growth, suggesting that Eph kinases function as tumor suppressors. Consistent with this notion, EphB2 expression is frequently lost or down-regulated in human colorectal cancer, and deficiency in EphB2 promoted colorectal tumorigenesis in APCMin/+ mice. Unlike EphB2, the function of which is lost or reduced in human cancer, several other Eph kinases are frequently overexpressed in human cancers. Elevated expression of EphA2 has been documented in various human cancers. While some in vitro studies support EphA2 as an oncoprotein, others are more consistent with anti-tumorigenic functions. Using EphA2 knockout mice, we recently found that deletion of EphA2 led to enhanced susceptibility to chemically-induced skin carcinogenesis with elevated tumor multiplicity and shortened latency. The latter studies show that EphA2 is a tumor suppressor gene in mammalian skin. Data presented here provide support for tumor suppressive functions of EphA2 in human prostate cancer cells, which could be exploited for cancer therapy. Much progress has been made in recent years in elucidating the bidirectional signal transduction of ephrins and Eph receptors. A1though the involvement of PI3-K pathway in EphA2 signaling has been documented in rat vascular smooth muscle cells and endothelial cells, the role of PI3-K pathway mediated by EphA2 receptor in tumor cells remains in question. Here we report that overexpression and activation of EphA2 negatively regulate Akt activities in prostate cancer cells. It has been well documented that Akt activity is regulated by PTEN and PI3-K expression and the Ser/Thr phosphatases such as PP2A or PP1. We found that Akt dephosphorylation induced by EphA2 activation was completely blocked by okadaic acid at 250 nM but not at 10 and 50 nM. Previous pharmacokinetics studies demonstrate that okadaic acid inhibits only PP2A at low concentrations (<100 nM), but both PP1 and PP2A at higher concentrations (>100 nM). These results indicate that EphA2 activation promotes Akt dephosphorylation at least in part through activation of the phosphatase PP1. Whether PTEN and PI3-K play a role is currently unknown and is under investigation. mTOR has become an attractive target for cancer therapy because of its key role as a mediator of the PI3-K/Akt signaling pathway. Recent cellular and biochemical studies show that the inhibition of mTOR by rapamycin in several different models causes activation of p-Akt (Ser473). We report here for the first time that ERK1/2 activities were also upregulated by rapamycin. The dual feedback activation of Akt and ERK1/2 by rapamycin in tumor cells may reduce its antitumor effects. Disruption of the feedback loops could enhance the antitumor effects of rapamycin. In support of this notion, rapamycin combined with the PI3-K inhibitor LY294002 or MEK1 inhibitor PD98059 exhibited enhanced inhibitory effects on the growth of human NSCLC cells and rapamycin-resistant ovarian cancer cell lines. In glioma cells, EGFR inhibitor EKI-785 blocked rapamycin-induced upregulation of Akt phosphorylation, and these cells displayed the synergistic antitumor effects of EKI-785 and rapamycin administration. We show here that EphA2 activation could simultaneously suppress Akt and ERK1/2 activities induced by rapamycin treatment. The dual inhibition could contribute to the cooperative growth inhibitory effects of rapamycin when used in combination with ephrin-A1. Collectively, the data presented here demonstrate that EphA2 kinase acts as a tumor suppressor in prostate cancer cell through, at leas in part, down-regulation of Akt and ERK1/2 kinase activities in our model. EphA2 activation and treatment with other cytostatic or cytotoxic agents may be a rational and useful strategy for the prevention and/or treatment of prostate cancer.

### Example 2

### Molecular Mechanisms of the Tumor Suppressor Function of EphA2 Kinase in Mammalian Lung

Lung cancer is the leading cause of cancer death in the US. In 2005 alone, 163,510 patients succumbed to lung cancer, more than the combined death rate from the cancers of breast, prostate, and colon-rectum (ACS, US Cancer Statistics, 2005). Despite a recent downward trend in death rate in the American men, lung cancer death in women and never smokers has continued to grow. Worldwide, the increasing consumption of tobacco products is assuring lung cancer epidemic at an alarming scale within the next decade. Existing strategies for lung cancer interventions have only marginally improved survival over the past several decades. Thus understanding the molecular mechanisms of lung cancer etiology and developing novel and mechanisms-based therapies constitute major challenges in cancer research today.

A majority of lung cancer is comprised of four main histologic subtypes, including small cell lung cancer (SCLC), and three non-small cell lung cancer (NSCLC) types including squamous cell carcinoma, adenocarcinoma, and large cell carcinoma. SCLC and squamous originate from major airways, while adenocarcinomas are more peripherally localized. Large cell carcinomas are the third type of NSCLC and are the less differentiated form of NSCLC. Together the three different NSCLC are responsible for 80% of all lung cancers, while SCLC make up the remaining 20%. Among NSCLC, adenocarcinoma is the most common type of lung cancer and is increasing rapidly worldwide. Intriguingly, adenocarcinomas differentially affect women and never smokers.

Similar to other human malignancies, lung cancer development is a multistage process resulting from cumulative genetic alterations. It is estimated that up to 10 genetic events are needed for a normal lung epithelial cell to become fully transformed. These changes can be grouped into two categories: the activation of oncogenes and the loss of tumor suppressor genes. Oncogenes that contribute to lung cancer include c-Myc, K-Ras, EGFR, Bc1-2 and cyclin-D1. Loss of tumor suppressor genes frequently involves Rb, p53 and p16. The list of lung cancer oncogenes and tumor suppressor genes as well as other modifier genes is likely to increase in the near future, because genome-wide screening in human and mouse has implicated numerous other loci. Interestingly, we have found that EphA2 receptor tyrosine kinase was a previously unrecognized lung tumor suppressor gene. Research on lung cancer has been facilitated by mouse model systems, which share remarkable morphologic, histologic and molecular features with human lung adenocarcinomas. Therefore, mouse models serve as valuable tools not only for understanding basic biology of but also for the development and validation of novel tumor intervention strategies. A range of mouse models for human lung cancer has been developed. These include spontaneous lung cancer in susceptible strains, and chemically-induced lung cancers using several carcinogens. Urethane, or ethyl carbamate, is a commonly used lung carcinogen that induces primarily adenocarcinomas, the most common types of lung cancer in human. As shown below, we found that EphA2 deletion led to increased susceptibility to urethane-induced lung carcinogenesis. The value of urethane carcinogenesis model has recently been validated molecularly, as tumors induced by urethane are found to share many key molecular signatures with human lung adenocarcinomas. Because carcinogen induction of primary lung tumorigenesis is highly reproducible in immune competent host, and simulates many steps in smoking-induced human lung cancer, it remains a valuable and powerful model system.

Transgenic and knockout mouse models where defined genetic lesions are introduced into the mouse genome have been generated that show increased lung tumorigenesis. This latter approach has recently been improved substantially by the generation of mouse strains carrying conditional oncogenes and tumor-suppressor genes allowing somatic induction of these mutations in a spatiotemporal fashion, thereby closely mimicking the sporadic character of human lung cancer. This invention utilizes some of the conditional model systems to investigate how EphA2 may regulate lung tumorigenesis.

Eph Receptor Tyrosine Kinases and their Ephrin Ligands

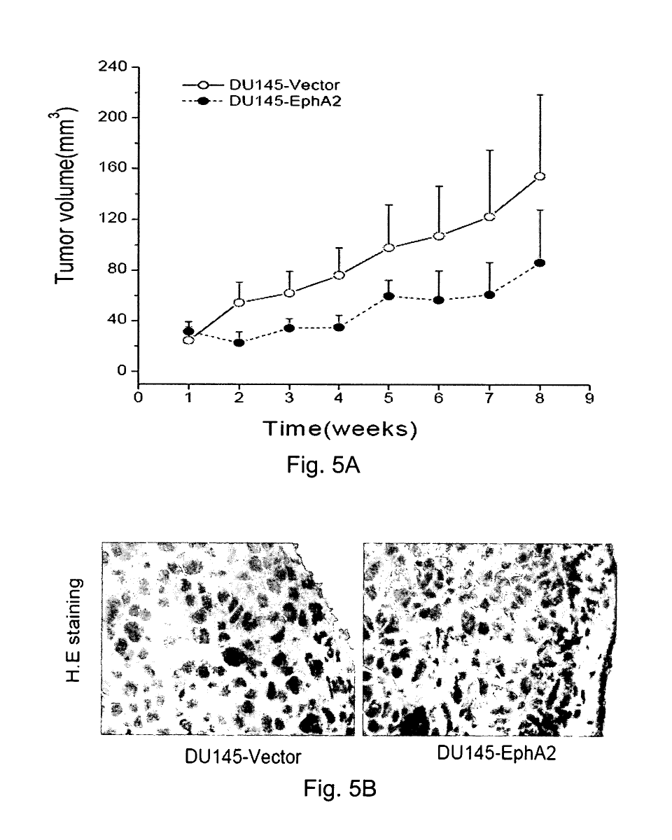

The first member of Eph subfamily of receptor tyrosine kinases (RTK) was cloned from an erythropoietin producing hepatoma cell line nearly two decades ago. Today, there are 14 Eph kinases in human genome, constituting 25% of all the known RTKs. They are divided into EphA and EphB kinases according to sequence homology and binding specificity of their membrane-anchored ligands called ephrins (FIG. 6). EphA kinases generally bind to GPI-anchored ephrin-As; EphB kinases preferentially target transmembrane ephrin-Bs. Two noted exceptions are EphA4 and EphB2 that cross-react with ephrin-B2 and ephrin-A5 respectively (FIG. 6). By binding to membrane-anchored ligands, Eph kinases mediate cell-cell contact signaling, and distinguish themselves from other receptor tyrosine kinases such as EGF receptor. Remarkably, Eph/ephrin signaling occurs in a bidirectional manner upon cell contact. Not only is there signaling by the Eph kinases (forward signaling), ephrins on the opposing cells are also capable of receptor-like signaling (reverse signaling).

Extensive early studies show that Eph kinase/ephrin interactions critically regulate the development of nervous and cardiovascular systems. In vitro and genetic evidences reveal that a primary consequence of Eph kinase activation is the repulsive guidance of migrating growth cones and neurons, although attractive adhesive responses have also been reported in some cases. Repulsive regulation of cell motility also plays an important role in the epithelial development. For example, EphB2/B3 and ephrinB1/B2 interactions control intestinal crypt morphogenesis in vivo. Negative regulation of cell motility has been suggested as one potential mechanism. Consistent with this suggestion, our in vitro studies showed that ephrin-B1 could potently inhibit colorectal epithelial cell motility in a Boyden chamber cell migration assay. Activation of EphA2 kinase similarly caused the collapse/retraction of HGF-induced invasive membrane protrusions on MDCK renal epithelial cells cultured in 3D matrix.

### Eph/Ephrin Bidirectional Signaling in Cancer

Despite first being cloned from a tumor cell line, the role of Eph kinases and ephrins in cancer remains poorly understood. Initial studies have shown abnormal expression of Eph and ephrins in various human tumors. These include overexpression of EphB6 in neuroblastoma, EphB4 in breast cancer, EphB2 in a variety of human tumors, and ephrin-A1 and ephrin-B2 in melanoma. Among EphA kinases, EphA2 is frequently overexpressed in variety of human malignancies (below). Perplexingly, earlier studies had shown that overexpression and activation of Eph kinases did not induce proliferative responses. This was in contrast with prototypic receptor tyrosine kinases, such as EGFR, that are known to promote cell proliferation when activated. Indeed, inventors were the first to report that EphA2 activation suppressed cell proliferation in some tumor cell types, further setting Eph kinases apart from prototypic RTKs.

More recently, Eph kinases have been found to be mutated in common human solid tumors. Mutational analysis of tyrosine kinome revealed somatic mutations in the kinase domain of EphA3 in human colorectal cancer. We found that the mutations caused the loss of EphA3 catalytic function. These loss-of-function mutations in EphA3 are more in line with tumor suppressor functions.

### EphA2 Kinase in Cancer: A Novel Tumor Suppressor

EphA2 was named epithelial cell kinase, or Eck, because of its wide distribution in epithelial cells in vitro and in vivo. Unlike EphB2, the function of which is lost or reduced in human cancer due to gene mutation or loss of expression, EphA2 is frequently overexpressed in human carcinomas including those of breast, lung, prostate, GI tract and kidney. This has led to the suggestion that EphA2 may be an oncoprotein. In one study, forced expression of EphA2 in MCF10A normal breast epithelial cells induced malignant transformation. Conversely, overexpression of dominant negative EphA2 reduced tumor growth and metastasis in 4T1 breast cancer xenografts in nude mice.

However, an increasing body of evidence shows that activation of endogenous EphA2 may have anti-tumorigenic functions. We reported previously that activation of endogenous EphA2 kinase in PC-3 cells reduced cell migration and proliferation. Similarly, in gastric cancer cells, where EphA2 is overexpressed, ephrin-A1 stimulation suppressed cell proliferation. Overexpression of EphA2 in MCF7 breast cancer and H1299 lung cancer cells led to spontaneous autophosphorylation and activation of EphA2, which resulted in decreased cell proliferation. Consistent with the growth suppressive effects of EphA2 activation, we found the Ras/ERK1/2 signaling cascade was inhibited upon ephrin-A1 stimulation of EphA2 in several different cell types. Moreover, EphA2 activation could antagonize EGF-, PDGF and VEGF-induced activation of the Ras/ERK1/2 signaling cascade. Down-regulation of ERK1/2 activities upon EphA2 ligation has subsequently been observed in HUVEC cells. Recently, a conditional feedback loop has been identified, whereby ERK activation promotes EphA2 expression, which in turn negatively regulates ERK kinase activities upon ligand stimulation. In contrast with these studies, it has been reported that ERK1/2 activation in tumor cells following EphA2 stimulation. In sum, existing literature using in vitro cell culture systems have led to divergent views on the role of EphA2 in regulating tumor cell signaling and behaviors.

Over the past several years, we have taken genetic approaches to systemically address this critical question. EphA2 knockout mice were used to investigate how EphA2 ablation may impact spontaneous as well as chemical-induced carcinogenesis. As detailed in preliminary results below, a series of discoveries has resulted from these studies. In aggregate, our data demonstrate that EphA2 is a previously unrecognized tumor suppressor gene. In a paper in press at Cancer Research, we reported that disruption of EphA2 led to dramatically increased susceptibility to tumor development in a classical DMBA/TPA two stage skin carcinogenesis model. Not only were there increased tumor multiplicity, shortened tumor latency, and accelerated growth rate, tumors arising in EphA2-null mice also displayed significantly enhanced malignant progression. The dramatically increased skin tumor susceptibility led us to suspect that EphA2-null mice could have become susceptible to spontaneous tumorigenesis. Indeed, careful examination of 14 month old mice revealed that about half of EphA2-null mice developed macroscopic and/or microscopic spontaneous lung tumors. Consistent with this observation, these mice were also much more susceptible to lung carcinogenesis induced by urethane, a well-characterized lung carcinogen. Therefore, EphA2 is a tumor suppressor gene in both mammalian skin and lung.

### Harnessing the Intrinsic Tumor Suppressor Function of EphA2 for Cancer Therapy

Activation of oncogenes and loss of tumor suppressor genes are two key underlying causes for tumor development and progression. The addiction of cancer cells to the function of oncogenes has made them attractive targets for cancer therapy, leading to the development of a variety of strategies that interrupt the function of oncogenes. An alternative strategy is to exploit the intrinsic anti-oncogenic properties of tumor suppressor genes. The power of tumor suppressor genes in controlling tumor development and progression is manifested by the fact that only one or a few cells in the human body will ever become a tumor, despite trillions of potential target cells. A significant challenge in targeting tumor suppressor genes is their loss of function by deletion or mutation. However, it is becoming evident that even in fully malignant tumor cells, some of the tumor suppressor genes remain intact but latent. Harnessing the innate tumor-suppressive gene function has emerged as a new approach in the fight against cancer. We propose that EphA2 represents one such gene that can be exploited therapeutically, because EphA2 is overexpressed on tumor cells in a latent form, which can be activated by its cognate ligands or other agonists to unleash its tumor suppressor activities.

Our preliminary studies demonstrate that in both mouse skin and lung tumors, EphA2 is upregulated compared with surrounding normal tissues, similar to what has been reported in human cancer. However, expression of ephrin-A1, a cognate ligand for EphA2, was frequently down-regulated, leading to reduced basal activation of EphA2. As a result, despite overexpression on the tumor cell surface, EphA2 is present in latent or partially active state. Consistent with this in vivo observation in mouse, in most human NSCLC cell lines, EphA2 was highly expressed, while ephrin-A1 was not. In vitro, stimulation of the EphA2 suppressed both ERK and Akt activities and inhibited cell migration and proliferation in NSCLC cells. Thus, ligand stimulation of the overexpressed but inactive EphA2 can unleash its latent tumor suppressor functions.

Because EphA2 ectodomain is readily accessible from the blood stream, we propose that the native ligands and other molecules that can bind to EphA2 and activate its tumor suppressor functions can be exploited for cancer therapy. We used the native ligand, ephrin-A1, to target EphA2 to investigate the molecular mechanism of the novel tumor suppressor function of EphA2, and to test whether EphA2 can be targeted for lung cancer therapy.

### Results

EphA2 disruption caused increased susceptibility to skin carcinogenesis. As discussed above, EphA2 receptor tyrosine kinase is frequently overexpressed in different human cancers. This has led to the suggestion that EphA2 may promote tumor development and progression, a notion supported by several in vitro and xenografts studies using human cell lines. However, evidence also exists that EphA2 possesses anti-tumorigenic properties, raising a critical question on the role of EphA2 kinase in tumorigenesis in vivo. Over the past two years, Inventors has taken genetic approaches to directly address this important question using EphA2 knockout mice. We reasoned that if EphA2 functions as an oncoprotein, ablation of EphA2 could lead to resistance to carcinogenesis. Conversely, if EphA2 is a tumor suppressor gene, EphA2-null could become more susceptible.

The model: To gain a proof-of-principle, we initially chose to use the classical DMBA/TPA two stage mouse skin chemical carcinogenesis model. This model has been widely utilized in assessing oncogene or tumor suppressor gene functions. It entails a single application of tumor initiation agent DMBA followed by twice weekly application of TPA as a tumor promoter. For this study, we obtained KST-085 line of EphA2 knockout mice established by a secretory trapping strategy. In this line, the secretory trapping retroviral vector was inserted at the boundary between exon 5 and intron 6, leading to the truncation of exons 6 to 17 encoding the second fibronectin type III repeat in the ectodomain all the way to the carboxyl terminus. The remaining ectodomain encoded by exons 1-5 is fused to neoR and β-gal (B-geo) reporter cassette under the control of EphA2 promoter, and is trapped inside the cells in secretory vesicles presumably in inactive form. The EphA2 knockout mice were originally on C57B1/6-129SvJ mixed genetic background known to be relatively resistant to DMBA/TPA-induced skin tumors. To improve the statistical power and to reduce latency period, we backcrossed the mice for four generations to FVB/NJ mice. Mice on FVB background have moderate susceptibility to carcinogenesis in the skin as well as other organ sites including the lung.

These skin carcinogenesis studies have provided the first proof-of-principle that EphA2 can function as a tumor suppressor in an epithelial tissue, three panels of data are presented here to highlight some of the key findings.

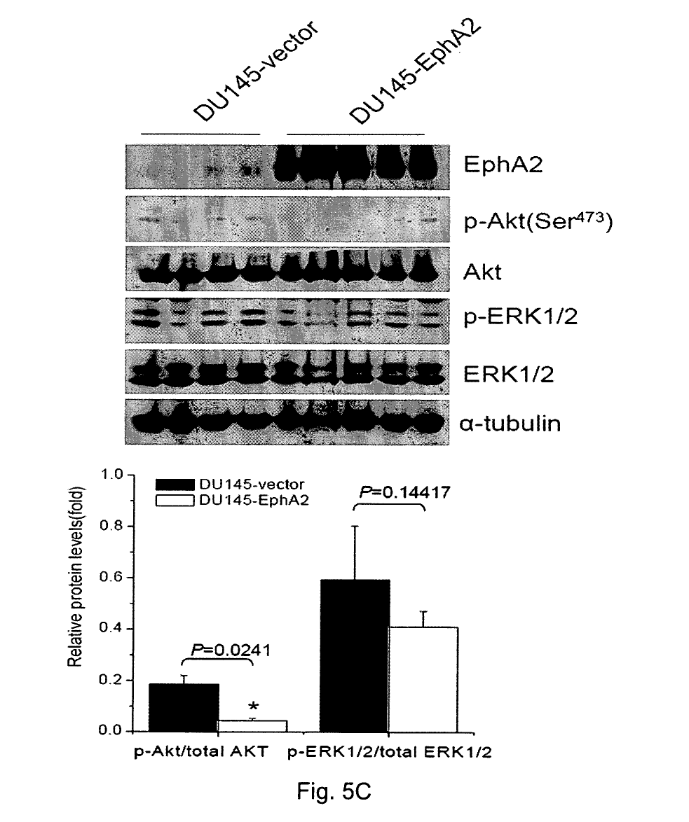

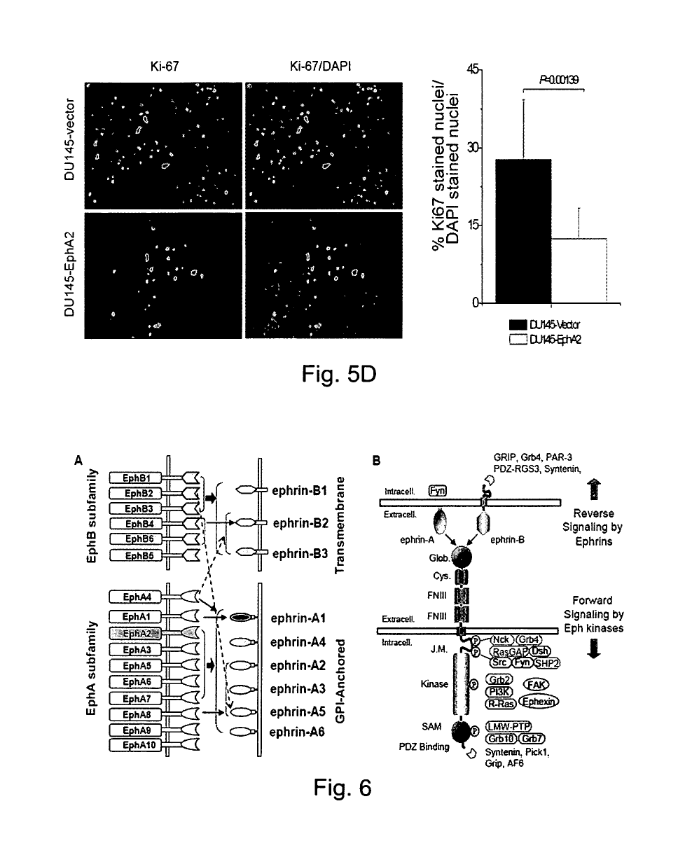

1) As shown in FIG. 7, deletion of EphA2 in mouse led to markedly enhanced susceptibility to skin carcinogenesis. EphA2-null mice developed skin tumors with an increased frequency and shortened latency (FIG. 7A,B). Moreover, tumors in homozygous knockout mice grew faster (FIG. 7C) and were twice more likely to show invasive malignant progression (FIG. 8).

2) Loss of EphA2 increased tumor cell proliferation, while apoptosis was not affected. These results are not shown here.

3) To determine the potential molecular mechanisms, primary A B keratinocytes were isolated. Treatment of primary keratinocytes from wild type mice with ephrin-A1, a ligand for EphA2, suppressed cell proliferation and inhibited ERK1/2 activities. Both effects were abolished in EphA2-null keratinocytes, suggesting that loss of ERK inhibition by EphA2 may be one of the contributing mechanisms for increased tumor susceptibility. In primary mouse embryonic fibroblasts (MEF), deletion of EphA2 also abolished ephrin-A1 stimulation-induced ERK1/2 inhibition. Moreover re-expression of EphA2 restored the inhibitory effects. Inventors were the first to report ERK1/2 suppression upon EphA2 activation. The data from primary keratinocytes and MEF cells demonstrate that EphA2 activation was necessary and sufficient to suppress ERK1/2 activities.

4) Interestingly, despite its tumor suppressive function, EphA2 was overexpressed in skin tumors compared with surrounding normal skin, similar to what has been reported in many human cancers. We propose that EphA2 overexpression may represent a compensatory feedback mechanism during tumorigenesis. This may be due to the activation of the Ras/ERK1/2 signaling cascade that has recently been shown to stimulate EphA2 expression, and mutated H-Ras is present in over 95% of all DMBA-induced skin tumors. Further discussion on this issue is given below. In summary, these results demonstrate that EphA2 is a previously unrecognized tumor suppressor gene in mammalian skin.

### EphA2 Knockout Mice Spontaneously Developed Lung Tumors and Showed Significantly Enhanced Susceptibility to Urethane-Induced Lung Carcinogenesis.

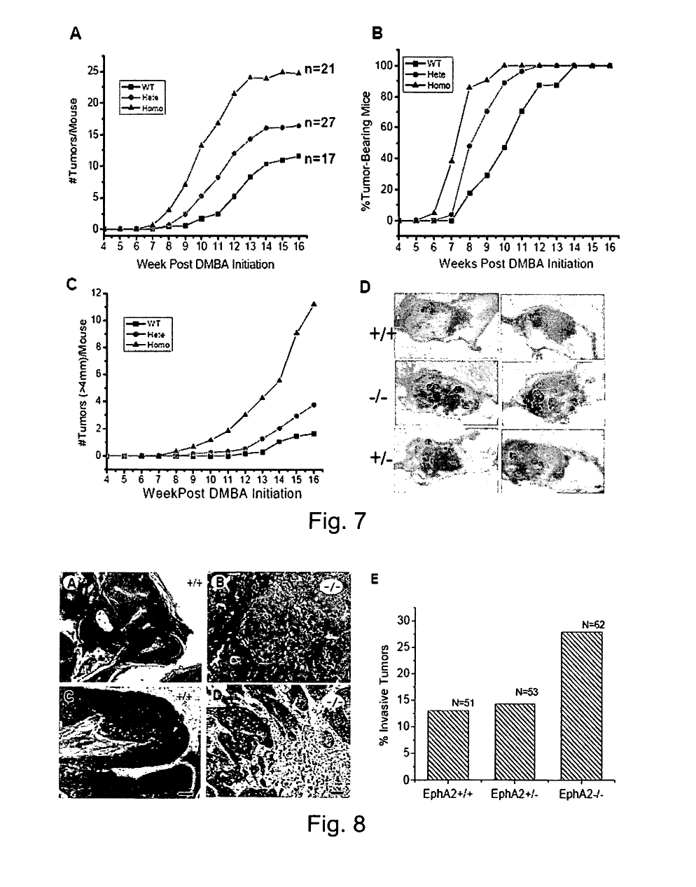

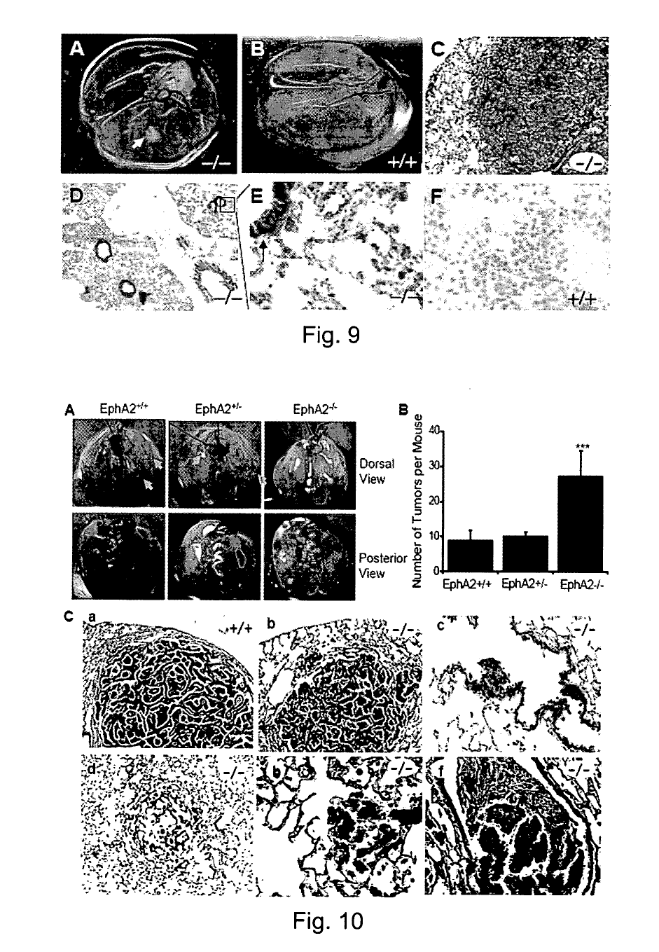

The dramatic increase in susceptibility to skin carcinogenesis in EphA2-null mice prompted us to investigate whether EphA2 knockout may predispose mice to spontaneous tumor development. Toward this end, mice were euthanized at 14 months of age and subject to detailed examination for evidence of spontaneous tumors. Excitingly, we found that 3 out of 11 EphA2−/− mice developed visible tumors on the surface of the lungs (FIG. 9 A,B). Histological analysis revealed one more mouse with internal macroscopic tumor, while 3 others had one to three microscopic lesions. A macroscopic adenoma is shown in FIG. 9C. In contrast, none of the 7 EphA2+/+ (FIG. 9B) and 10 EphA2+/−littermates had lung tumor development at this age, suggesting that EphA2 is a lung tumor suppressor gene. Consistent with a tumor suppressor role of EphA2 in the lung, staining of EphA2-null mice for beta-geo knockin reporter cassette shoes that EphA2 kinase is actively expressed in the lung. (FIG. 9D, E). The highest expression was observed in trachea and bronchiole epithelia, while lower expression level was observed in alveolar cells (FIG. 9D,E). Although multiple lines of Eph kinase and ephrin knockout mice have been reported in the literature, this was the first demonstration of spontaneous tumorigenesis resulting from a single Eph kinase knockout. Since no macroscopic neoplastic lesions were detected in other organ sites including the skin, EphA2 appears to preferentially affect lung tumorigenesis.

This is a major reason that we chose to pursue the studies on the role of EphA2 in lung cancer. Next, we subjected EphA2 knockout mice to the well-established lung carcinogenesis model using urethane as a carcinogen. A cohort of 8 weeks old mice were injected with 1 mg/kg urethane. Twenty weeks later, mice were sacrificed and surface lung tumors were enumerated. 100% of mice from all three genotypes developed lung tumors. Consistent with spontaneous lung tumorigenesis, we found that EphA2-null mice on FVB genetic background developed an average 29 lung surface tumors compared with about 8 tumors in wild type littermates, representing a three fold increase in tumor multiplicity (FIG. 10A,B) (p<0.001). There was also a trend of increase in EphA2+/−mice, although the difference was statistically insignificant. As expected, most tumors in wild type and knockout mice at 20 weeks were adenomas (FIG. 10C; a,b). Serial sections also revealed the presence of preneoplastic lesions, including epithelial hyperplasia (EH) in small airways, atypical adenomatous hyperplasia (AAH), and microadenomas. Interestingly, we noticed, while a majority of tumors from both wild type and EphA2-null mice arose in alveolar tissues, more EH and intra-bronchiolar adenomas developed in EphA2 knockout mice (22%) than in wild type mice (7%).

In sum, the development of spontaneous lung tumors in EphA2-null mice and their significantly elevated susceptibility to chemically-induced lung carcinogenesis show that EphA2 is a novel lung tumor suppressor gene. Genetic studies using inbred mice with different susceptibilities to chemically-induced lung carcinogenesis have led to the identification of multiple loci linked to lung tumor susceptibility, echoing the increasing genetic complexities of human lung cancer development. However, the identities for many of genes involved are yet to be determined. Intense efforts are currently under way to molecularly characterize these genes using various strategies including bioinformatics and positional cloning, because of the obvious importance of such efforts in understanding the etiology and therapeutic intervention of lung cancer. The finding that EphA2 is a novel lung tumor suppressor gene may aid in these efforts and broaden our view on the network of lung tumor suppressor genes. Moreover, the unique expression pattern and mode of action of EphA2 as detailed below make it an attractive target for therapeutic intervention of lung cancer, which remains by far the deadliest human malignancy.

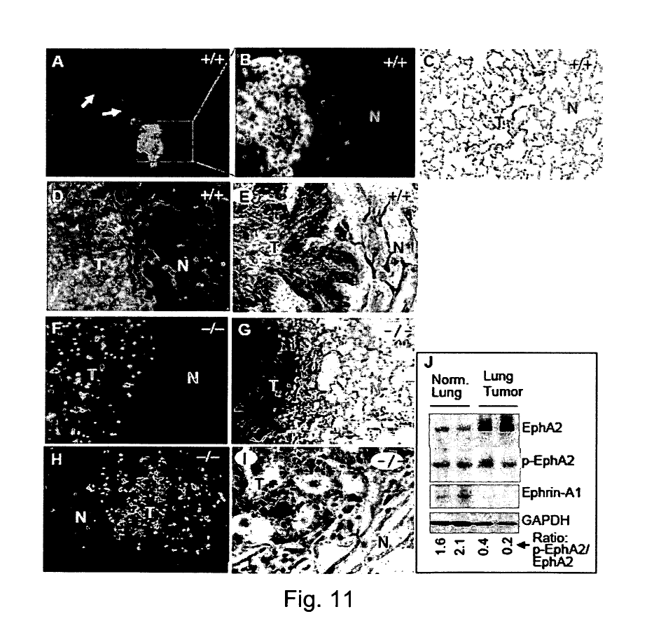

EphA2 was overexpressed from very early stages of lung carcinogenesis in wild type mice, bu was present in a partially active or latent form. Similar to EphA2 overexpression in various human cancers as well as in DMBA/TPA-induced mouse skin tumors, we found that EphA2 was significantly upregulated in most of the urethane-induced tumors examined compared with surrounding normal lung in wild type animals (FIG. 11,AE). Even in the very early microadenomas and atypical adenomatous hyperplasia (AAH), EphA2 was substantially overexpressed (FIG. 11A,B,C). In normal lung, EphA2 was most highly expressed in bronchiole epithelium (FIG. 9), but the expression levels in the tumors far exceeded that adjacent bronchiole (FIG. 11A, arrow). Biochemical analyses confirmed upregulation of EphA2 in lung tumors compared with surrounding normal lung (FIG. 11J).

In EphA2 knockout mice, part of the ectodomain of EphA2 was fused to reporter cassette and trapped inside the cells in inactive form. We took advantage of this fact and stained EphA2−/−tumors with an anti-EphA2 ectodomain antibody. We found that the truncated EphA2 was also upregulated in both spontaneous (FIG. 11F) and urethane-induced tumor tissues (FIG. 11H,I). Note the intracellular localization of the truncated EphA2 in knockout tumors compared with membrane localization in wild type tumors, e.g., compare FIGS. 11E and I). Thus, the overexpression of both wild type EphA2 and truncated EphA2/β-geo cassette in tumors suggest that EphA2 is likely to be transcriptionally upregulated during spontaneous and chemically-induced tumorigenesis.

Next we determined the activation status of EphA2 by immunoblotting with an antibody against the phospho-specific antibody raised by Inventors that recognizes the activated form of Eph kinases. We found that the relative degree of EphA2 activation was significantly reduced in tumors; the ratio between active and total EphA2 dropped from around 1.8 to 0.3 (FIG. 11J). The expression of ephrin-A1, a major cognate ligand for EphA2 in the lung, was reduced in tumors compared with normal tissue (FIG. 11I). Loss of ligand expression may contribute to reduced activation level of EphA2, although other mechanisms cannot be ruled out. In sum, the overexpressed EphA2 in lung tumors exists in a functionally latent state (FIG. 11I).

It was demonstrated that EphA2 is a direct transcription target of the Ras/Raf/MEK/ERK1/2 signaling cascade. Expression of oncogenic Raf enhanced EphA2 expression in NIH-3T3 fibroblasts. We have similarly found that transformation of NRP152 normal rat prostatic epithelial cells with the activated Ras or Raf oncogene caused a several fold increase in EphA2 expression. Since Ras is activated in most urethane-induced and spontaneous lung tumors, we propose that the upregulated EphA2 may represent a compensatory feedback mechanism against oncogenic transformation in cells that have the activated Ras. It should be noted that the Ras/ERK1/2 signaling pathway can be activated by a variety of mechanisms in addition to Ras activating mutations, including the acquisition of the autocrine loop involving GPCRs or growth factor receptors.

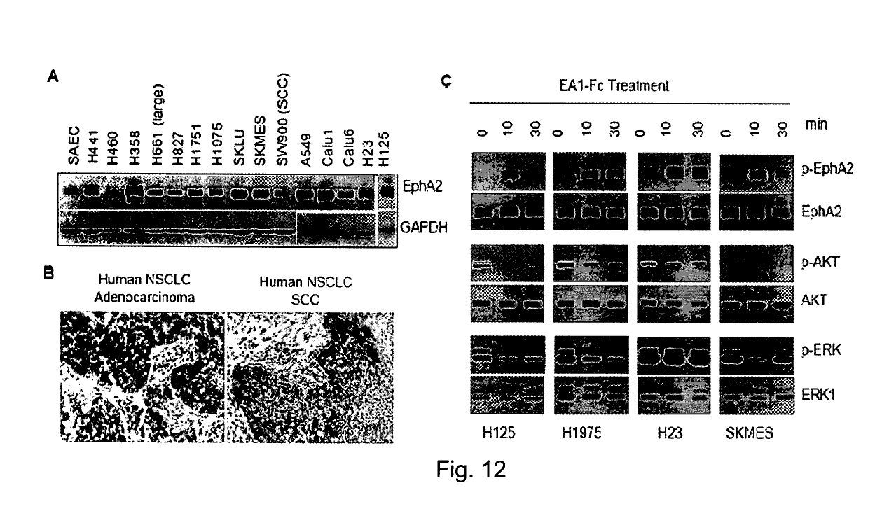

A major cause for high mortality and poor therapeutic response in lung cancer patients is the lack of early detection and intervention. By the time of diagnosis, most lung cancers have already reached late stage and metastasized. The striking upregulation of EphA2 in early lesions including microadenomas and AAH leads us to hypothesize that EphA2 can serve as an early marker of lung cancer development and target for therapeutic intervention, which will be tested in this proposal. EphA2 was overexpressed in human NSCLC cells in vitro and in vivo, and ligand stimulation of EphA2 led to simultaneous inhibition of Akt and ERK1/2 kinase activities. Having shown that EphA2 functions as a tumor suppressor gene in mouse, we examined the expression and function of EphA2 in human NSCLC cell lines. We found that moderate to high levels of EphA2 were expressed in most of the NSCLC cell lines examined (FIG. 12A). EphA2 has previously been shown to be overexpressed in human lung cancer specimens. Here we verified these observations and detected high levels of EphA2 expression in different histological types of human NSCLC cancer specimens. Examples of an adenocarcinoma and a large cell carcinoma are shown in FIG. 62B. Thus, in both primary human specimens in vivo and cell lines in vitro, EphA2 was found to be overexpressed in human NSCLC cells. A1though EphA2 was overexpressed on most NSCLC cell lines, under resting conditions there was very little basal activation of its catalytic activities, since a phospho-specific antibody raised in Inventors against the activated form of Eph kinases detected no or very low signal (FIG. 12C, time 0). However, addition of an exogenous ligand, ephrin-A1-Fc, to the culture medium led to rapid and strong activation of EphA2, suggesting that EphA2 was in latent but functionally intact form. We reported previously that EphA2 activation can inhibit the Ras/ERK1/2 signaling cascade in several different cell types. Moreover, studies using primary mouse keratinocytes and MEF cells showed that EphA2 was necessary and sufficient to mediate ERK1/2 inhibition upon ligand stimulation. Here we show that ERK1/2 activities were also inhibited in a subset of human NSCLC cells examined (FIG. 12C). More interestingly, we found that Akt activities were also suppressed in these cells (FIG. 12C). In H23 cells, EphA2 activation selectively inhibited Akt but not ERK1/2, suggesting that distinct mechanisms may be involved in Akt vs. ERK1/2 inhibition. Constitutive activation of Ras/ERK1/2 and/or PI3K/Akt signaling pathways represents major underlying causes of tumor progression as well as therapeutic resistance. The simultaneous inhibition of ERK and Akt by EphA2 activation in lung cancer cells suggest that the latent EphA2 kinase activities can be harnessed for lung cancer therapy.

Ligand Stimulation of EphA2 Inhibited Human NSCLC Cell Migration and Proliferation, which was Further Enhanced by EphA2 Overexpression.

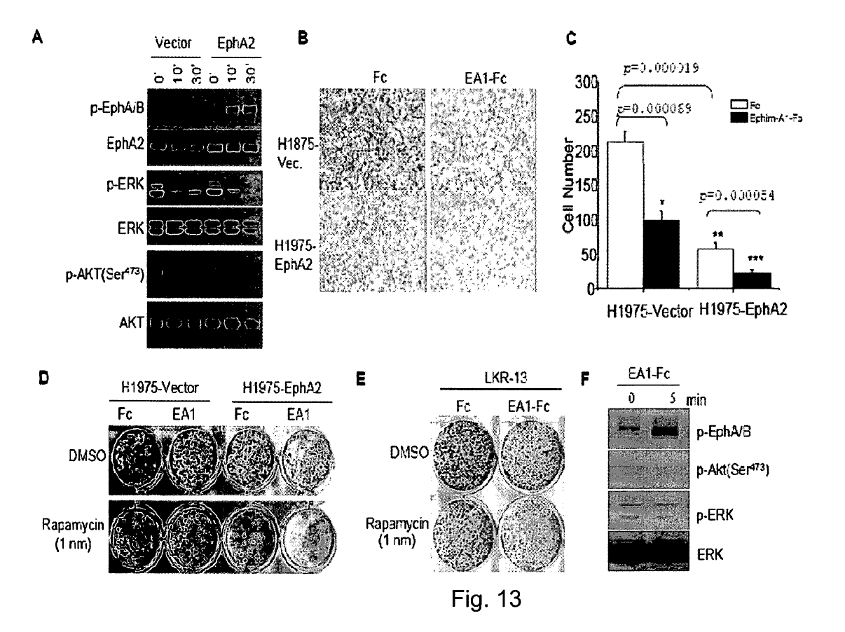

To examine the functional significance of EphA2 upregulation, we overexpressed EphA2 in H1975 NSCLC cells that express moderate levels of endogenous EphA2, and subjected the vector control and EphA2-overexpressing cells to migration and proliferation assays in the presence or absence of ligand stimulation. These studies led to the following observations: 1) Compared with vector control cells, EphA2 overexpression further promoted the inhibitory effects of ephrin-A1 stimulation on ERK1/2 and Akt activities (FIG. 13A). 2) Consistent with our early report, ephrin-A1 stimulation suppressed integrin dependent cell migration toward fibronectin in parental and vector control cells; the effects were enhanced by EphA2 overexpression (FIG. 13B,C). 3) In a clonal growth assay, ephrin-A1 stimulation suppressed the proliferation of vector control cells (FIG. 13D, top left two wells). EphA2-overexpressing cells showed reduced basal cell proliferation (FIG. 13D, compare first and third wells in top row), and were more responsive to ephrin-A1 stimulation-induced growth inhibition (FIG. 13D, top row).

Rapamycin Cotreatment with Ephrin-A1 had Cooperative Inhibitory Effects on Cell Proliferation, which was Sensitized by EphA2 Overexpression.

The PI3K/Akt/mTOR pathway is frequently activated in various human malignancies through mutations in pathway components or activation of upstream signaling molecules such as EGF and IGF-1 receptors. Rapamycin and its derivatives are now actively being evaluated in clinical trials. We found that Rapamycin treatment could inhibit the growth of vector control H1975 cells, and the effects were significantly enhanced by co-treatment with ephrin-A1 (FIG. 13D, H1975 vector). Moreover, EphA2 overexpression markedly sensitized cells to growth inhibition by Rapamycin; co-treatment with ephrin-A1 further augmented the effects, leading to near complete arrest of cell growth (FIG. 13D, H1975-EphA2). Cellular and biochemical analysis revealed that the reduced cell proliferation but not increased apoptosis was responsible for the effects of ephrin-A1 (not shown). We conclude that ephrin-A1 and Rapamycin cotreatment could additively or synergistically suppress proliferation of lung cancer cells. Our data also indicate that EphA2 expression level may predict lung cancer responsiveness to Rapamycin therapy.

Mouse Lung Cancer Cells Derived from Tumors Harboring Activated K-Ras were Sensitive to Growth Inhibition by Ephrin-A1, Rapamycin or their Combination.

Our data suggest that EphA2 may counteract the transforming effects of K-ras oncogene, at least in part by suppressing Akt activation by the activated Ras. To test this, we directly investigated whether EphA2 activation can inhibit the growth of cells known to be caused by constitutively active K-Ras. We chose a recently described mouse lung cancer cell line LKR-13 derived from a tumor induced by K-ras. FIG. 13E shows that stimulation of LKR-13 cells with ephrin-A1 or Rapamycin suppressed cell growth, and cotreatment with both further increased the inhibitory effects. Activities of Akt were suppressed by ephrin-A1 in these cells upon ephrin-A1 stimulation. Therefore, we propose that lung tumors harboring constitutively active K-Ras can be targeted for therapeutic intervention with ephrin-A1, Rapamycin or their combination.

### Systemic Administration of Ephrin-A1 led to Tumor Homing and EphA2 Activation.

In the last submission, we proposed that the latent EphA2 kinase overexpressed on lung tumor cells could be targeted by systemically administered ephrin-A1-Fc. A series of in vivo experiments since then has demonstrated the feasibility of the approach. These are described below.

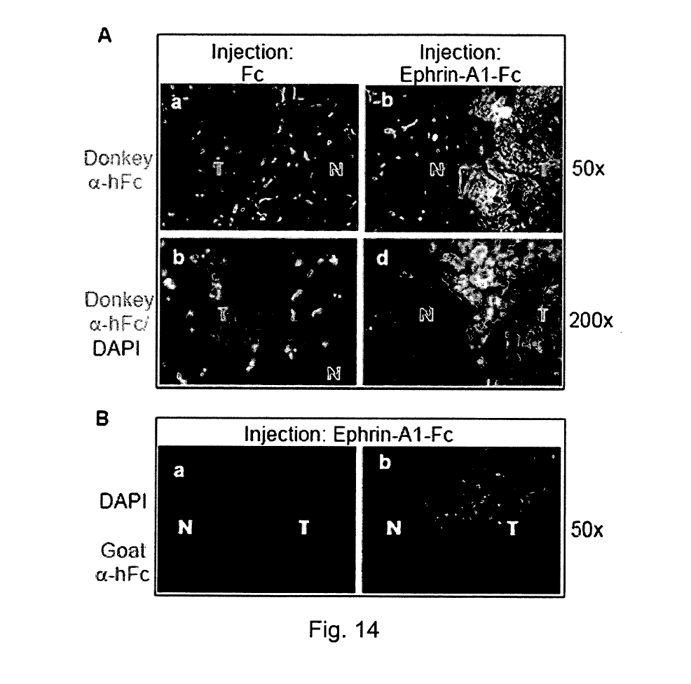

First, we tested if systemically administered ephrin-A1-Fc could home to urethane-induced lung tumors that overexpress EphA2. Lung tumors were induced in wild type mice with urethane. Twenty-five weeks later, mice were injected i.p. with 5 μg/g ephrin-A1-Fc. Fc fusion partner alone was used as control. Two hours after injection, mice were sacrificed, and tumors were dissected out; frozen sections were subject to immunofluorescence staining to detect whether there was a selective homing of ephrin-A1-Fc to tumor tissues relative to control Fc. FIG. 14 shows ephrin-A1-Fc preferentially homed to tumor tissues. Both FITC-conjugated donkey anti-Fc and Texas-Red-conjugated goat anti-human Fc antibodies yielded similar patterns of selective tumor homing.

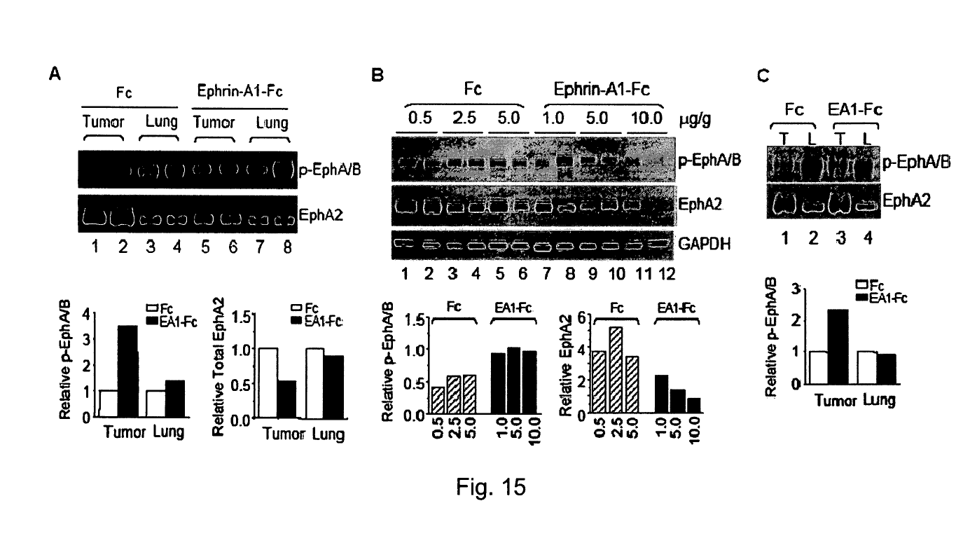

If ephrin-A1 indeed homed to the tumor through binding to the overexpressed EphA2, it was predicted that EphA2 would become activated. Therefore, we examined activation status of EphA2 following Fc or ephrin-A1-Fc injection. PBS alone was used as another control, which yielded the same results as Fc (not shown). Tumor tissues and adjacent normal lung were harvested under dissection microscope and lysed. EphA2 kinase was immunoprecipitated, and immunoblotted for EphA2 activation status with our phospho-specific antibody that recognizes the activated Eph kinases. Consistent with earlier observations, we found EphA2 was highly activated in normal lungs, while EphA2 in tumor tissues were poorly activated despite much higher levels of expression (FIG. 15A, lanes 1-4). Systemic administration of ephrin-A1 caused an increased EphA2 activation in tumors (FIG. 15A). EphA2 in normal lungs, on the other hand, was not significantly affected, presumably because EphA2 kinase in normal tissues was already ligated by endogenous ligand. Similar to most other receptor tyrosine kinases, ligand induced activation of EphA2 kinase leads to receptor endocytosis and degradation. We show here that EphA2 overexpressed on tumor cells in vivo also became degraded following systemic treatment with ephrin-A1-Fc.

An ephrin-A1-Fc dose-response study was carried next. Evidence of EphA2 activation was detected even at 1 μg/g (FIG. 15B, lane 8). Increasing doses of ephrin-A1 caused further activation of EphA2 kinase as indicated by increased degradation of the receptor. Indeed, at the highest dose tested (10 μg/g), most of EphA2 kinase was degraded (FIG. 15B, lane 11,12). However, after long-term treatment, EphA2 expression did come back. Prolonged ephrin-A1 treatment has been reported to stimulate EphA2 expression, which may account for the recovery of the EphA2 expression. Alternatively, the tumor cells might have become desensitized following prolonged exposure to ephrin-A1-Fc. Notably, even after prolonged treatment, EphA2 still showed elevated activation, compared with Fc control. Due to the lack of sufficient number of mice in the pilot studies, we were not able to perform statistically meaningful experiments on the effects of ephrin-A1 treatment on tumor growth. However, the pilot experiments did provide strong support for the feasibility of the studies proposed in Aim 3 designed to investigate whether ephrin-A1 can inhibit tumor growth in vivo.

In summary, systemically administered ephrin-A1-Fc can selectively home to tumors, and causes the activation of dormant EphA2 kinase overexpressed on urethane-induced autochthonous lung tumors.

### Systemic Administration of Ephrin-A1 Inhibited the Growth of Human NSCLC Cell Xenograft

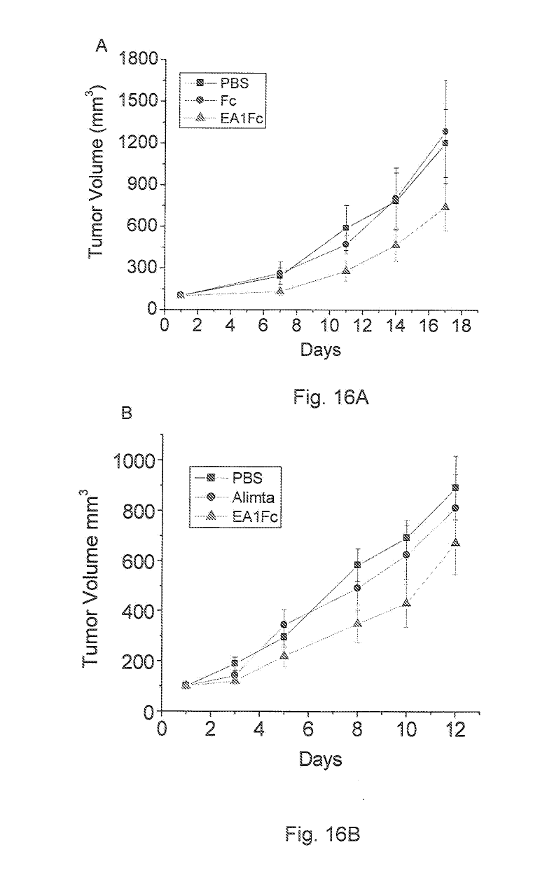

To directly test the feasibility of targeting EphA2 for lung cancer therapy, we systemically treated nude mice bearing human NSCLC xenografts with ephrin-A1-Fc. H1975 cells were implanted into the hind flanks of 8 week-old nude mice at 2×106 cells/site. One week later, when tumors had reached 100 mm3, ephrin-A1-Fc was injected i.p. twice a week at 5 μg/g body weight. FIG. 16A shows that treatment with ephrin-A1-Fc, but not Fc control, significantly inhibited the growth of H1975 xenograft.

We next compared ephrin-A1-Fc and Alimta/Pemetrexed for therapeutic efficacy against H460 xenograft. H460 cells are derived from a lung adenocarcinoma that express moderate levels of EphA2 (not shown). Alimta or Pemetrexed is an antifolate that inhibits thymidylate synthase, dihydrofolate reductase, and glycinamide ribonucleotide formyl transferase, enzymes necessary for purine and pyrimidine synthesis, thus causing cell-cycle arrest in the S phase. Alimta has been approved for clinical treatment for NSCLC. As shown in FIG. 16B, ephrin-A1-Fc treatment significantly suppressed the growth of H460 xenograft, while Alimta alone has minimal effects.

Therefore, ephrin-A1-Fc could inhibit the growth of both H1975 and H460 xenografts that harbor constitutively active EGFR and K-ras, respectively. These results are consistent with in vitro observations (FIG. 13) and strongly support the feasibility of harnessing the intrinsic tumor suppressor activities of EphA2 for lung cancer therapy.

Establishment of Cell Lines from EphA2+/+, EphA2+/−, and EphA2−/− Lung Tumors, which are Tumorigenic in FVB Syngeneic Host

We reasoned that mechanistic studies of the tumor suppressor functions of EphA2 kinase would significantly benefit from the establishment of immortalized cell lines from tumors of defined genotypes. For example, the EphA2-null background will provide a valuable platform to dissect signaling pathways implicated in tumor suppression. The mechanistic studies on EphA2 kinase have been hampered by the fact that most epithelial cells express various levels of the kinase. Because even very low levels of EphA2 can effectively mediate cell signaling, RNAi knockdown may not be an ideal approach in evaluating mechanisms of EphA2 kinase function. The availability of EphA2-null cell lines will significantly alleviate this problem.

Isolation of murine lung tumor cell lines is known to be a technically challenging task. The LKR-13 used in FIG. 16E,F was established by using primary mouse embryonic fibroblast (MEF) as feeder layer, which is widely used in cultivating stem cells. We adapted this new strategy and successfully isolated multiple lung tumor cell lines, which were further characterized as illustrated in FIG. 16.

Most of the cell lines displayed epithelial morphology (FIG. 16A, phase). Staining for β-geo reporter cassette and EphA2 revealed the expected expression patterns (FIG. 16A). All cell lines were also stained positive for keratins (FIG. 16A), confirming their epithelial origin. The cell lines were then stimulated with ephrin-A1-Fc for up to two hours and subject to immunoblot analyses. EphA2 kinase in WT and heterozygous cells had very low basal activation status. Ligand treatment potently and rapidly stimulated its activation (FIG. 16B). In contrast, EphA2 homozygous KO cells showed little change in p-EphA/B signal. Because the our phospho-EphA/B antibody was raised against the conserved phospho-dityrosine motifs in the juxtamembrane region, it should recognize most members of activated Eph kinases. The lack of increased p-EphA/B signal suggests that there was very low level expression of other members of EphA subfamily kinases in the EphA2-null cells, which provides an “clean” system for studying EphA kinase signaling. Similar to human NSCLC cells, there was a sustained inhibition of Akt activities in EphA2+/+ and EphA2+/− cells (FIG. 16B). In contrast, ERK1/2 kinase activities were minimally affected, which may reflect K-ras activation in all three cell lines (not shown). Strikingly, the basal Akt activities in EphA2−/− cells were much lower than those in EphA2+/+ and EphA2+/− cells, despite the prediction to the contrary. The reasons are uncertain; one possible explanation is that homozygous EphA2 deletion may enable the lung cells to bypass the requirement for Akt activation during malignant transformation in the early stages of tumorigenesis.

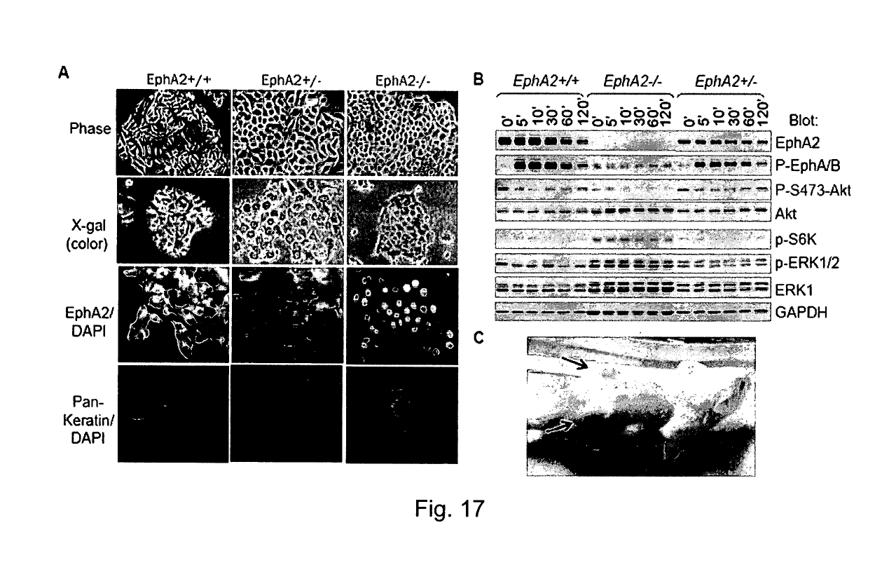

During the cause of our studies, we have backcrossed the EphA2 KO mice on C57BL/6-129SvJ mixed genetic background to FVB/NJ mice for 12 generations, effectively generating congenic EphA2 KO mice on the inbred FVB/NJ background. The mice provide a syngeneic host for the lung tumor-derived cell lines. This system allows us to investigate some important questions that cannot be addressed in nude mice, including whether the tumor suppressor function of EphA2 is host-dependent or cell-autonomous. In an exciting proof of principle, we found that an EphA2-null lung tumor cell line was highly tumorigenic in FVB mice, yielding tumors in about two weeks after s.c. cell implantation (FIG. 17C). Rapid tumor development was also observed upon injection of the cells to nude mice (not shown).

Those skilled in the art will recognize, or be able to ascertain using no more than routine experimentation, numerous equivalents to the compounds and methods of use thereof described herein. Such equivalents are considered to be within the scope of this invention and are covered by the following claims. All patents, publications, and references cited in the present application are herein incorporated by reference in their entirety.

1. A method of treating a neoplastic disorder in a subject comprising:
administering to an Eph kinase expressing neoplastic cell of the subject being treated a therapeutically effective amount of an EphA agonist and an mTOR inhibitor.

2. The method of claim 1, wherein the administration of the EphA agonist modulates an EphA kinase in the cell of the subject.

3. The method of claim 2, wherein modulating comprises activating Eph kinase in the cell of the subject.

4. The method of claim 1, wherein the neoplastic disease comprises at least one of lung cancer, brain cancer, prostate cancer or breast cancer.

5. The method of claim 4, wherein the cancer is associated with expression of Eph kinase in the cancerous cells.

6. (canceled)

7. The method of claim 1, wherein the mTOR inhibitor comprises sirolimus.

8. The method of claim 1, wherein the mTOR inhibitor is a sirolimus analog.

9. The method of claim 1, wherein the EphA agonist is an ephrin.

10. The method of claim 9, wherein the ephrin is ephrin-A1, ephrin A5, or a combination thereof.

11. The method of claim 1, wherein the EphA agonist has a cooperative effect with the mTOR inhibitor.

12. The method of claim 2, wherein the Eph kinase is selected from the group consisting of EphA1, EphA2, or EphA3 or a combination thereof.

13. A method of treating cancer in a subject comprising:
administering to an Eph kinase expressing cancer cell of the subject being treated a therapeutically effective amount of an EphA agonist and an mTOR inhibitor.

14. The method of claim 13, wherein the administration of the EphA agonist modulates an EphA kinase in the cancer cell of the subject.

15. The method of claim 13, wherein the cancer comprises at least one of lung cancer, brain cancer, prostate cancer or breast cancer.

16. The method of claim 13, wherein the mTOR inhibitor comprises sirolimus.

17. The method of claim 13, wherein the mTOR inhibitor is a sirolimus analog.

18. The method of claim 13, wherein the EphA agonist is an ephrin.

19. The method of claim 18, wherein the ephrin is ephrin-A1, ephrin A5, or a combination thereof.

20-24. (canceled)
